SEQUENCE LISTING (14 sequences)

SEQ ID NO: 1 - DNA, 573 bp, Artificial Sequence
  PD1 wild-type domain
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaaggcccagatcaaagagagcctgagagccgagctgagagtgaccgagagaagggccgaagtgcctaccgcccaccctagcccatctccaagacctgccggccagttccagacactggtcgtgggagtcgtgggcggactgctgggatctctggtgctgctcgtgtgggtgctggccgtgatt

SEQ ID NO: 2 - DNA, 465 bp, Artificial Sequence
  PD1 (A99L) domain
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaagctccagatcaaagagagcctgcgggcagagctcagggtgacagagagaagggcagaagtgcccacagcccac

SEQ ID NO: 3 - DNA, 240 bp, Artificial Sequence
  CD28 intracellular domain
tgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 4 - DNA, 888 bp, Artificial Sequence
  MyD88 domain
atggctgctggcggacctggcgctggatctgctgctcctgtgtctagcaccagcagcctgcctctggccgccctgaatatgagagtgcggcggagactgagcctgttcctgaacgtgcggacacaggtggccgccgattggacagctctggccgaggaaatggacttcgagtacctggaaatccggcagctggaaacccaggccgaccctacaggacgcctgctggatgcttggcagggcagaccaggcgcttctgtggggagactgctggaactgctgaccaagctgggccgggacgacgtgctgctggaactgggccctagcatcgaagaggactgccagaagtacatcctgaagcagcagcaggaagaggccgagaagcctctgcaggtggcagccgtggatagcagcgtgccaagaacagctgagctggccggaatcaccaccctggacgatcctctgggccacatgcccgagagattcgacgccttcatctgctactgccccagcgacatccagttcgtgcaggaaatgatcagacagctggaacagaccaactaccggctgaagctgtgcgtgtccgaccgggatgtgctgcctggcacctgtgtgtggtctatcgccagcgagctgatcgagaagcggtgcagacggatggtcgtggtggtgtccgacgactacctgcagtccaaagagtgcgacttccagaccaagttcgccctgagcctgagccctggcgcccaccagaagagactgatccccatcaagtacaaggccatgaagaaagagttccccagcatcctgcggttcatcaccgtgtgcgactacaccaacccctgcaccaagtcctggttctggaccagactggccaaggccctgtctctgcct

SEQ ID NO: 5 - DNA, 348 bp, Artificial Sequence
  CD2 domain
aagcggaagaagcagcggagcagaagaaacgacgaggaactggaaaccagagcccacagagtggccaccgaggaaagaggcagaaagccccaccagatccccgcctccacccctcagaatcctgccacctctcagcaccctccacctcccccaggacacagaagccaggcccctagccatagacctccaccacccggacaccgggtgcagcatcagcctcagaaaagaccccctgcccctagcggcacacaggtgcaccagcagaaaggccccccactgcccagacctagagtgcagcctaagcctcctcacggcgctgccgagaatagcctgagcccctccagcaac

SEQ ID NO: 6 - DNA, 705 bp, Artificial Sequence
  PD1(WT)CD28
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccactgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 7 - DNA, 1470 bp, Artificial Sequence
  PD1(WT)MYD88
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaaggcccagatcaaagagagcctgagagccgagctgagagtgaccgagagaagggccgaagtgcctaccgcccaccctagcccatctccaagacctgccggccagttccagacactggtcgtgggagtcgtgggcggactgctgggatctctggtgctgctcgtgtgggtgctggccgtgattggcggaggaatggctgctggcggacctggcgctggatctgctgctcctgtgtctagcaccagcagcctgcctctggccgccctgaatatgagagtgcggcggagactgagcctgttcctgaacgtgcggacacaggtggccgccgattggacagctctggccgaggaaatggacttcgagtacctggaaatccggcagctggaaacccaggccgaccctacaggacgcctgctggatgcttggcagggcagaccaggcgcttctgtggggagactgctggaactgctgaccaagctgggccgggacgacgtgctgctggaactgggccctagcatcgaagaggactgccagaagtacatcctgaagcagcagcaggaagaggccgagaagcctctgcaggtggcagccgtggatagcagcgtgccaagaacagctgagctggccggaatcaccaccctggacgatcctctgggccacatgcccgagagattcgacgccttcatctgctactgccccagcgacatccagttcgtgcaggaaatgatcagacagctggaacagaccaactaccggctgaagctgtgcgtgtccgaccgggatgtgctgcctggcacctgtgtgtggtctatcgccagcgagctgatcgagaagcggtgcagacggatggtcgtggtggtgtccgacgactacctgcagtccaaagagtgcgacttccagaccaagttcgccctgagcctgagccctggcgcccaccagaagagactgatccccatcaagtacaaggccatgaagaaagagttccccagcatcctgcggttcatcaccgtgtgcgactacaccaacccctgcaccaagtcctggttctggaccagactggccaaggccctgtctctgcct

SEQ ID NO: 8 - DNA, 930 bp, Artificial Sequence
  PD1(WT)CD2
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaaggcccagatcaaagagagcctgagagccgagctgagagtgaccgagagaagggccgaagtgcctaccgcccaccctagcccatctccaagacctgccggccagttccagacactggtcgtgggagtcgtgggcggactgctgggatctctggtgctgctcgtgtgggtgctggccgtgattggcggaggaaagcggaagaagcagcggagcagaagaaacgacgaggaactggaaaccagagcccacagagtggccaccgaggaaagaggcagaaagccccaccagatccccgcctccacccctcagaatcctgccacctctcagcaccctccacctcccccaggacacagaagccaggcccctagccatagacctccaccacccggacaccgggtgcagcatcagcctcagaaaagaccccctgcccctagcggcacacaggtgcaccagcagaaaggccccccactgcccagacctagagtgcagcctaagcctcctcacggcgctgccgagaatagcctgagcccctccagcaac

SEQ ID NO: 9 - DNA, 705 bp, Artificial Sequence
  PD1(A99L)CD28
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaagctccagatcaaagagagcctgcgggcagagctcagggtgacagagagaagggcagaagtgcccacagcccactgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 10 - DNA, 1470 bp, Artificial Sequence
  PD1(A99L)MYD88
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcgtgggagtcgtgggcggactgctgggatctctggtgctgctcgtgtgggtgctggccgtgattggcggaggaatggctgctggcggacctggcgctggatctgctgctcctgtgtctagcaccagcagcctgcctctggccgccctgaatatgagagtgcggcggagactgagcctgttcctgaacgtgcggacacaggtggccgccgattggacagctctggccgaggaaatggacttcgagtacctggaaatccggcagctggaaacccaggccgaccctacaggacgcctgctggatgcttggcagggcagaccaggcgcttctgtggggagactgctggaactgctgaccaagctgggccgggacgacgtgctgctggaactgggccctagcatcgaagaggactgccagaagtacatcctgaagcagcagcaggaagaggccgagaagcctctgcaggtggcagccgtggatagcagcgtgccaagaacagctgagctggccggaatcaccaccctggacgatcctctgggccacatgcccgagagattcgacgccttcatctgctactgccccagcgacatccagttcgtgcaggaaatgatcagacagctggaacagaccaactaccggctgaagctgtgcgtgtccgaccgggatgtgctgcctggcacctgtgtgtggtctatcgccagcgagctgatcgagaagcggtgcagacggatggtcgtggtggtgtccgacgactacctgcagtccaaagagtgcgacttccagaccaagttcgccctgagcctgagccctggcgcccaccagaagagactgatccccatcaagtacaaggccatgaagaaagagttccccagcatcctgcggttcatcaccgtgtgcgactacaccaacccctgcaccaagtcctggttctggaccagactggccaaggccctgtctctgcct

SEQ ID NO: 11 - DNA, 930 bp, Artificial Sequence
  PD1(A99L)CD2
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcgtgggagtcgtgggcggactgctgggatctctggtgctgctcgtgtgggtgctggccgtgattggcggaggaaagcggaagaagcagcggagcagaagaaacgacgaggaactggaaaccagagcccacagagtggccaccgaggaaagaggcagaaagccccaccagatccccgcctccacccctcagaatcctgccacctctcagcaccctccacctcccccaggacacagaagccaggcccctagccatagacctccaccacccggacaccgggtgcagcatcagcctcagaaaagaccccctgcccctagcggcacacaggtgcaccagcagaaaggccccccactgcccagacctagagtgcagcctaagcctcctcacggcgctgccgagaatagcctgagcccctccagcaac

SEQ ID NO: 12 - DNA, 63 bp, Artificial Sequence
  PD1tm
gtgggagtcgtgggcggactgctgggatctctggtgctgctcgtgtgggtgctggccgtgatt

SEQ ID NO: 13 - DNA, 9 bp, Artificial Sequence
  GLY3 linker
ggcggagga

SEQ ID NO: 14 - DNA, 390 bp, Artificial Sequence
  Inducible synthetic promoter, iSynPro
ctgcttagggttaggcgttttgcgctgcttcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccat

(19) United States
(12) Patent Application Publication
(10) Pub. No.: US 2023/0374104 A1
(43) Pub. Date: Nov. 23, 2023

(54) METHODS AND COMPOSITIONS COMPRISING PD1 CHIMERIC POLYPEPTIDES

(71) Applicant: Seattle Children's Hospital (dba Seattle Children's Research Institute), Seattle, WA (US)

(72) Inventors: Michael C. Jensen, Seattle, WA (US); Adam Johnson, Seattle, WA (US); James Rosser, Seattle, WA (US)

(21) Appl. No.: 18/030,476

(22) PCT Filed: Oct. 7, 2021
(86) PCT No.: PCT/US2021/053971

§ 371 Date: Apr. 5, 2023

Related U.S. Application Data
(60) Provisional application No. 63/089,752, filed on Oct. 9, 2020.

(51) Int. Cl. C07K 14/705; C07K 14/725

(52) U.S. Cl. CPC C07K 14/70596; C07K 14/7051

(57) ABSTRACT

Some embodiments of the methods and compositions provided herein relate to chimeric proteins comprising a programmed cell death protein 1 (PD 1) extracellular domain and an intracellular immunostimulatory domain. Some embodiments include a cell containing a PD 1 chimeric polypeptide and a chimeric antigen receptor (CAR). In some embodiments, the CAR comprises a ligand binding domain capable of specifically binding to a target antigen on a solid tumor. More embodiments relate to therapies to treat, inhibit or ameliorate certain disorders, such as a cancer, such as a solid tumor.

### CROSS-REFERENCE TO RELATED APPLICATIONS

This application claims priority to U.S. Prov. App. No. 63/089,752 filed Oct. 9, 2020 entitled “METHODS AND COMPOSITIONS COMPRISING PD1 CHIMERIC POLYPEPTIDES,” which is expressly incorporated by reference in its entirety.

### REFERENCE TO SEQUENCE LISTING

The present application is being filed along with a Sequence Listing in electronic format. The Sequence Listing is provided as a file entitled SCR1254WOSEQLIST, created Oct. 4, 2021, which is approximately 13 Kb in size. The information in the electronic format of the Sequence Listing is incorporated herein by reference in its entirety.

### FIELD OF THE INVENTION

Some embodiments of the methods and compositions provided herein relate to chimeric proteins comprising a programmed cell death protein 1 (PD1) extracellular domain and an intracellular immunostimulatory domain. Some embodiments include a cell containing a PD1 chimeric polypeptide and a chimeric antigen receptor (CAR). In some embodiments, the CAR comprises a ligand binding domain configured to specifically bind to a target antigen on a solid tumor. More embodiments relate to therapies to treat, inhibit or ameliorate certain disorders, such as a cancer, such as a cancer presenting one or more solid tumors.

### BACKGROUND OF THE INVENTION

In cell-based adoptive immunotherapy, T cells isolated from a patient can be modified to express synthetic proteins that enable the cells to perform new therapeutic functions after they are subsequently transferred back into the patient. Examples of such synthetic proteins are chimeric antigen receptors (CARs) and engineered T cell Receptors (TCR). An example of a currently used CAR is a fusion of an extracellular recognition domain (e.g., an antigen-binding domain), a transmembrane domain, and one or more intracellular signaling domains. Upon antigen engagement, the intracellular signaling portion of the CAR can initiate an activation-related response in an immune cell, such as release of cytolytic molecules to induce tumor cell death. However, there is a continued need for improved cell-based adoptive immunotherapies.

### SUMMARY OF THE INVENTION

Some embodiments of the methods and compositions provided herein include a polynucleotide encoding a chimeric polypeptide comprising: an extracellular PD1 domain; and an intracellular immunostimulatory domain.

In some embodiments, the extracellular PD1 domain comprises an A99L substitution. In some embodiments, the extracellular PD1 domain comprises a polypeptide encoded by a nucleotide sequence having at least 95%, 96%, 97%, 98%, 99%, or 100% sequence identity to SEQ ID NO:01 or 02.

In some embodiments, the intracellular immunostimulatory domain is selected from an intracellular domain of a protein selected from myeloid differentiation primary response 88 protein (MYD88), CD28, or CD2. In some embodiments, the intracellular immunostimulatory domain comprises a polypeptide encoded by a nucleotide sequence having at least 95%, 96%, 97%, 98%, 99%, or 100% sequence identity with any one of SEQ ID NOs:03-05. In some embodiments, the intracellular immunostimulatory domain comprises MYD88 or CD2.

In some embodiments, the PD1 domain is linked to the intracellular immunostimulatory domain via a transmembrane domain. In some embodiments, the transmembrane domain comprises a PD1 transmembrane domain. In some embodiments, the PD1 transmembrane domain is encoded by the nucleotide sequence of SEQ ID NO:12.

In some embodiments, the transmembrane domain is linked to the intracellular immunostimulatory domain via a linker. In some embodiments, the linker is encoded by the nucleotide sequence of SEQ ID NO: 13.

In some embodiments, the chimeric polypeptide is encoded by a nucleotide sequence having at least 95%, 96%, 97%, 98%, 99%, or 100% sequence identity with any one of SEQ ID NOs:06-12.

Some embodiments also include a nucleic acid encoding a cell surface selectable marker. In some embodiments, the cell surface selectable marker is selected from a truncated HER2 (Her2tG) polypeptide, a truncated EGFR (EGFRt) polypeptide, or a truncated CD19 (CD19t).

In some embodiments, a nucleic acid encoding the chimeric polypeptide and the nucleic acid encoding a cell surface selectable marker are linked via a ribosomal skip sequence. In some embodiments, the ribosomal skip sequence is selected from P2A, T2A, E2A or F2A.

Some embodiments also include a constitutive promoter operably linked to a nucleic acid encoding the chimeric polypeptide. In some embodiments, the promoter comprises an E1a promoter.

Some embodiments also include an inducible promoter operably linked to a nucleic acid encoding the chimeric polypeptide. In some embodiments, the inducible promoter comprises the nucleotide sequence set forth in SEQ ID NO:14.

Some embodiments also include an inducible cytotoxic gene. In some embodiments, the cytotoxic gene encodes a protein selected from a thymidine kinase, thymidine kinase fused to thymidylate kinase, oxidoreductase, deoxycytidine kinase, uracil phosphoribosyltransferase, cytosine deaminase, or cytosine deaminase fused to uracil phosphoribosyltransferase. In some embodiments, the cytotoxic gene encodes a thymidine kinase.

Some embodiments also include a nucleic acid encoding a chimeric antigen receptor (CAR).

Some embodiments include a vector comprising any one of the foregoing polynucleotides. In some embodiments, the vector comprises a viral vector. In some embodiments, the vector is selected from a lentiviral vector, an adeno-associated viral vector, or an adenoviral vector. In some embodiments, the vector comprises a lentiviral vector.

Some embodiments include a polypeptide encoded by any one of the foregoing polynucleotides.

Some embodiments include a cell comprising any one of the foregoing polynucleotides.

Some embodiments also include a fourth nucleic acid encoding a chimeric antigen receptor (CAR), or a CAR protein. In some embodiments, the CAR is capable of specifically binding to a target antigen expressed by a cancer cell. In some embodiments, the target antigen comprises a solid tumor cell surface antigen. In some embodiments, the target antigen comprises CD171.

In some embodiments, the cell is a natural killer cell. In some embodiments, the cell is a T cell. In some embodiments, the cell is derived from a CD4+ T cell, a CD8+ T cell, a precursor T cell, or a hematopoietic stem cell. In some embodiments, the CD8+ T cell is a CD8+ cytotoxic T lymphocyte cell selected from the group consisting of a naïve CD8+ T cell, a central memory CD8+ T cell, an effector memory CD8+ T cell, and a bulk CD8+ T cell. In some embodiments, the CD4+ cell is a CD4+ helper T lymphocyte cell selected from the group consisting of a naïve CD4+ T cell, a central memory CD4+ T cell, an effector memory CD4+ T cell, and a bulk CD4+ T cell.

In some embodiments, the cell is a primary cell. In some embodiments, the cell is mammalian. In some embodiments, the cell is human. In some embodiments, the cell is ex vivo.

Some embodiments include a pharmaceutical composition comprising any one of the foregoing cells and a pharmaceutically acceptable excipient.

Some embodiments include a method of treating, ameliorating or inhibiting a disorder in a subject, comprising: administering any one of the foregoing cells to the subject in need thereof, wherein the cell comprises a chimeric antigen receptor (CAR); optionally wherein said subject is selected or identified to receive a medicament for said disorder, such as by clinical or diagnostic evaluation for the presence of said disorder. In some embodiments, the disorder comprises a cancer comprising a target antigen, wherein the CAR is capable of specifically binding to the target antigen. In some embodiments, the cancer comprises a solid tumor. In some embodiments, the cancer is selected from colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma. In some embodiments, the cell is autologous to the subject. In some embodiments, the cell is not autologous to the subject In some embodiments, the subject is mammalian. In some embodiments, the subject is human.

Some embodiments include any one of the foregoing cells for use in treating, ameliorating or inhibiting a disorder, such as a cancer in a subject.

Some embodiments include use of any one of the foregoing cells as a medicament, such as for use in treating, ameliorating or inhibiting a disorder, such as a cancer in a subject.

Some embodiments include a method of preparing an effector cell, comprising introducing a polynucleotide of any one of the foregoing polynucleotides into a cell.

Some embodiments include a method of preparing an effector cell having an enhanced cytotoxic activity, comprising: introducing a polynucleotide of any one of the foregoing polynucleotides into a cell, wherein the effector cell comprises a chimeric antigen receptor (CAR) capable of specifically binding to a target antigen, and wherein the effector cell has an enhanced cytotoxic activity in the presence of the target antigen compared to a cell lacking the polynucleotide. In some embodiments, the enhanced cytotoxic activity comprises an increased level of cytokine expression, and/or an increased level of cytolysis of a target cell expressing the target antigen. In some embodiments, the cytokine is selected from IL-2, IFN-gamma, TNF-alpha, or granzyme B.

Some embodiments include a method of inhibiting a target cell expressing a target antigen, comprising contacting the target cell with an effector cell comprising a polynucleotide of any one of the foregoing polynucleotides, wherein the effector cell comprises a chimeric antigen receptor (CAR) capable of specifically binding to the target antigen.

In some embodiments, the target antigen is a cancer antigen. In some embodiments, the target antigen comprises a solid tumor cell surface antigen. In some embodiments, the target antigen comprises CD171, CD19, or an EGFR.

In some embodiments, the effector cell is a natural killer cell. In some embodiments, the effector cell is a T cell. In some embodiments, the effector cell is a CD4+ T cell, a CD8+ T cell, a precursor T cell, or a hematopoietic stem cell. In some embodiments, the CD8+ T cell is a CD8+ cytotoxic T lymphocyte cell selected from the group consisting of a naïve CD8+ T cell, a central memory CD8+ T cell, an effector memory CD8+ T cell, and a bulk CD8+ T cell; and the CD4+ cell is a CD4+ helper T lymphocyte cell selected from the group consisting of a naïve CD4+ T cell, a central memory CD4+ T cell, an effector memory CD4+ T cell, and a bulk CD4+ T cell. In some embodiments, the effector cell is a primary cell. In some embodiments, the effector cell is mammalian. In some embodiments, the effector cell is human. In some embodiments, the effector cell is ex vivo.

### BRIEF DESCRIPTION OF THE DRAWINGS

FIG. 1 is a schematic diagram of certain polynucleotides encoding PD1 chimeric polypeptides.

FIG. 2 depicts a flow cytometry analysis of CD8+ T cells for a Her2tG marker co-expressed with a certain PD1 chimeric polypeptide including: a PD1(A99L):CD28 polypeptide; a PD1(wild type):CD28 polypeptide; a PD1(A99L):MYD88 polypeptide; a PD1(wild type):MYD88 polypeptide; a PD1(A99L):CD2 polypeptide; or a PD1(wild type):CD2 polypeptide.

FIG. 3 depicts a bar graph for an interferon 7 (IFN-g) release assay with effector cells and target cells. Effector cells included: CD8+ T cells expressing a certain PD1 chimeric polypeptide; and target cells included: K562 cells, K562 cells expressing OKT3, or K562 cells expressing OKT3 and PD-L1.

FIG. 4 depicts a bar graph for an interleukin-2 (IL-2) release assay with effector cells and target cells. Effector cells included: CD8+ T cells expressing a certain PD1 chimeric polypeptide; and target cells included: K562 cells, K562 cells expressing OKT3, or K562 cells expressing OKT3 and PD-L1.

FIG. 5 depicts a bar graph for a tumor necrosis factor-α (TNF-α) release assay with effector cells and target cells. Effector cells included: CD8+ T cells expressing a certain PD1 chimeric polypeptide; and target cells included: K562 cells, K562 cells expressing OKT3, or K562 cells expressing OKT3 and PD-L1.

FIG. 6 depicts line graphs for a chromium release assay with effector cells and target cells. Effector cells included: CD8+ T cells expressing a certain PD1 chimeric polypeptide co-expressed with a Her2tG marker polypeptide; and target cells included: K562 cells, K562 cells expressing OKT3, or K562 cells expressing OKT3 and PD-LL.

FIG. 7 depicts a flow cytometry analysis of CD8+ T cells for either a Her2tG marker co-expressed with a certain PD1 chimeric polypeptide, or an EGFRt marker co-expressed with an 806 chimeric antigen receptor (CAR).

FIG. 8 depicts line graphs for a chromium release assay with effector cells and target cells. Effector cells included: CD8+ T cells expressing a certain PD1 chimeric polypeptide and an 806 CAR; and target cells included: K562 cells, K562 cells expressing OKT3, K562 cells expressing OKT3 and PD-L1, K562 cells expressing EGFRvIII, and K562 cells expressing EGFRvIII and PD-L1.

FIG. 9 depicts a bar graph for an interleukin-2 (IL-2) release assay with effector cells and target cells. Effector cells included: CD8+ T cells expressing a certain PD1 chimeric polypeptide and an 806 CAR; and target cells included: K562 cells, K562 cells expressing OKT3, K562 cells expressing OKT3 and PD-L1, K562 cells expressing EGFRvIII, and K562 cells expressing EGFRvIII and PD-L1.

FIG. 10 depicts a bar graph for an interferon γ (IFN-g) release assay with effector cells and target cells. Effector cells included: CD8+ T cells expressing a certain PD1 chimeric polypeptide and an 806 CAR; and target cells included: K562 cells, K562 cells expressing OKT3, K562 cells expressing OKT3 and PD-L1, K562 cells expressing EGFRvIII, and K562 cells expressing EGFRvIII and PD-L1.

FIG. 11 depicts a bar graph for a tumor necrosis factor-α (TNF-α) release assay with effector cells and target cells. Effector cells included: CD8+ T cells expressing a certain PD1 chimeric polypeptide and an 806 CAR; and target cells included: K562 cells, K562 cells expressing OKT3, K562 cells expressing OKT3 and PD-L1, K562 cells expressing EGFRvIII, and K562 cells expressing EGFRvIII and PD-L1.

FIG. 12 depicts a flow cytometry analysis of H9 cells containing a 806 CAR and a PD1 chimeric polypeptide.

FIG. 13A depicts flow analysis against MESF beads for PD1 chimeric polypeptides.

FIG. 13B depicts flow analysis against beads for PD1 chimeric polypeptides.

FIG. 13C depicts a graph for a F-P ratio for PD1 chimeric polypeptides.

FIG. 14 depicts a graph for relative affinity of PD1-L1 for PD1 chimeric polypeptides.

FIG. 15 depicts a seahorse XF cell mitochondria stress test profile.

FIG. 16 depicts a graph of oxygen consumption rate for CD8+ T cells from 3 donors (D1, D2, and D3) containing the 806 CAR, and a PD1 chimeric polypeptide.

FIG. 17A depicts a graph of oxygen consumption rate for CD8+ T cells from D1 containing the 806 CAR, and a PD1 chimeric polypeptide.

FIG. 17B depicts a graph of oxygen consumption rate for CD8+ T cells from D2 containing the 806 CAR, and a PD1 chimeric polypeptide.

FIG. 17C depicts a graph of oxygen consumption rate for CD8+ T cells from D3 containing the 806 CAR, and a PD1 chimeric polypeptide.

FIG. 18 depicts a graph for weighted score at day 14 with regard to ‘Be2-CD19t tumor’ and to weighted score for S1Sp1 in which circles represent rank order of transgenes in CD4+ cells, while triangles represent CD8+ cells.

FIG. 19 depicts a time-line for a study to examine the effects on effector CD8 T cells containing either anti-CD19 CAR alone, or anti-CD19 CAR and a PD1 chimeric polypeptide, challenged with target cells expressing a truncated CD19 polypeptide in a first challenge and a second challenge

FIG. 20A depicts concentrations of IL-2 or TNF-alpha in supernatants from co-cultured target and effector cells at day 2, from the study shown in FIG. 19.

FIG. 20B depicts concentrations of interferon-gamma and granzyme B in supernatants from co-cultured target and effector cells at day 2, from the study shown in FIG. 19.

FIG. 21A depicts concentrations of IL-2 or TNF-alpha in supernatants from co-cultured target and effector cells at day 4, from the study shown in FIG. 19.

FIG. 21B depicts concentrations of interferon-gamma and granzyme B in supernatants from co-cultured target and effector cells at day 4, from the study shown in FIG. 19.

### DETAILED DESCRIPTION

Some embodiments of the methods and compositions provided herein relate to chimeric proteins comprising a programmed cell death protein 1 (PD1) extracellular domain and an intracellular immunostimulatory domain. Some embodiments include a cell containing a PD1 chimeric polypeptide and a chimeric antigen receptor (CAR). In some embodiments, the CAR comprises a ligand binding domain capable of or configured to specifically bind to a target antigen on a solid tumor. More embodiments relate to therapies to treat, inhibit or ameliorate certain disorders, such as a cancer, such as a cancer that presents one or more solid tumors.

Immunotherapy using T cells containing chimeric antigen receptors (CARs) has seen great success in treating liquid tumors; however, such therapies can be limited in therapies to treat solid tumors. For example, T cells containing CARs express on their surface programmed cell death protein 1 (PD1). Cells within a solid tumor may upregulate PD-L1, a ligand for PD1. An interaction between PD1 expressed on the surface of a CAR T cell and the PD-L1 can result in CAR T cell inhibition, loss of effector function, and cellular dysfunction. Such potential shortcomings of CAR T cell therapy in solid tumors necessitates a need to augment the CAR T cell therapy. The use of PD1 chimeric polypeptides encoded by transgenes is an approach that can overcome the short coming of the existing CAR T cell technology.

In some embodiments, PD1 chimeric polypeptides comprise two components to switch from a normally immunoinhibitory signal to an immunostimulatory signal. A receptor portion of the protein comprises an extracellular PD1 domain and is responsible for the interaction with PD1 ligands, such as PD-L1. Engagement of endogenous PD1 with PD-L1 would normally result in an immunoinhibitory signal. By replacing the intracellular portion of an endogenous PD1 with an immunostimulatory protein domain a PD1 chimeric polypeptide can switch prior inhibitory interactions with ligands, such as PD-L1, to stimulatory interactions. In some embodiments, the intracellular domain of the PD1 chimeric polypeptide can include a TLR adaptor protein, such as MYD88, a costimulatory protein such as CD2 or CD28.

In some embodiments, the extracellular PD1 domain can be modified to have enhanced activity compared with a PD1 chimeric polypeptide comprising a wild-type PD1 extracellular domain. In some embodiments, the extracellular PD1 domain can include a A99L mutation. For example, the modified extracellular PD1 domain can result in a cell containing the PD1 chimeric polypeptide having enhanced binding with a ligand, such as PD-L1, and/or enhanced intracellular signaling events. In some embodiments, a cell containing a PD1 chimeric polypeptide and a CAR can have enhanced activities compared to a cell lacking the PD1 chimeric protein.

Some of the methods and compositions provided herein can include aspects disclosed in Prosser M.E., et al. (2012) Mol Immunol. 51:263-72 which is expressly incorporated by reference in its entirety.

### Definitions

Unless defined otherwise, all technical and scientific terms used herein have the same meaning as commonly understood by one of ordinary skill in the art to which the invention pertains.

As used herein, “a” or “an” may mean one or more than one.

“About” as used herein when referring to a measurable value is meant to encompass variations of ±20% or ±10%, more preferably ±5%, even more preferably ±1%, and still more preferably ±0.1% from the specified value.

As used herein, “nucleic acid” or “nucleic acid molecule” have their plain and ordinary meaning in view of the whole specification and may to refer to, for example, polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, or exonuclease action. Nucleic acid molecules can be composed of monomers that are naturally-occurring nucleotides (such as DNA or RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars or carbocyclic sugar analogs. Examples of modifications in a base moiety include alkylated purines or pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some embodiments, a nucleic acid sequence encoding a fusion protein is provided.

As used herein, “coding for” or “encoding” has its plain and ordinary meaning when read in light of the specification, and includes, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

As used herein, “chimeric antigen receptor” (CAR) has its plain and ordinary meaning when read in light of the specification, and may include but is not limited to, for example, a synthetically designed receptor comprising a ligand binding domain of an antibody or other protein sequence that binds to a molecule associated with a disease or disorder and is, preferably, linked via a spacer domain to one or more intracellular signaling domains of a cell, such as a T cell, or other receptors, such as one or more costimulatory domains. Chimeric receptor can also be referred to as artificial cell receptors or T cell receptors, chimeric cell receptors or T cell receptors, chimeric immunoreceptors, or CARs. These receptors can be used to graft the specificity of a monoclonal antibody or binding fragment thereof onto a cell, preferably a T-cell, with transfer of their coding sequence facilitated by viral vectors, such as a retroviral vector or a lentiviral vector. CARs can be, in some instances, genetically engineered T cell receptors designed to redirect T cells to target cells that express specific cell-surface antigens. T cells can be removed from a subject and modified so that they can express receptors that can be specific for an antigen by a process called adoptive cell transfer. The T cells are reintroduced into the patient where they can then recognize and target an antigen. CARs are also engineered receptors that can graft an arbitrary specificity onto an immune receptor cell. CARs are considered by some investigators to include the antibody or antibody fragment, preferably an antigen binding fragment of an antibody, the spacer, signaling domain, and transmembrane region. Due to the surprising effects of modifying the different components or domains of the CAR described herein, such as the epitope binding region (for example, antibody fragment, scFv, or portion thereof), spacer, transmembrane domain, and/or signaling domain), the components of the CAR are frequently distinguished throughout this disclosure in terms of independent elements. The variation of the different elements of the CAR can, for example, lead to a desired binding affinity, such as a stronger binding affinity for a specific epitope or antigen.

The CARs graft the specificity of a monoclonal antibody or binding fragment thereof or scFv onto a T cell, with the transfer of their coding sequence facilitated by vectors. In order to use CARs as a therapy for a subject in need, a technique called adoptive cell transfer is used in which T cells are removed from a subject and modified so that they can express the CARs that are specific for an antigen. The T cells, which can then recognize and target an antigen, are reintroduced into the patient.

As used herein, a “ribosome skip sequence” has its plain and ordinary meaning when read in light of the specification, and includes, for example, a sequence that during translation, forces the ribosome to “skip” the ribosome skip sequence and translate the region after the ribosome skip sequence without formation of a peptide bond. Several viruses, for example, have ribosome skip sequences that allow sequential translation of several proteins on a single nucleic acid without having the proteins linked via a peptide bond. As described herein, this is the “linker” sequence. In some alternatives of the nucleic acids provided herein, the nucleic acids comprise a ribosome skip sequence between the sequence for the chimeric antigen receptor and the sequence of the marker protein, such that the proteins are co-expressed and not linked by a peptide bond. In some embodiments, the ribosome skip sequence is a P2A, T2A, E2A or F2A sequence.

As used herein, a “marker sequence,” has its plain and ordinary meaning when read in light of the specification, and includes, for example, a protein that is used for selecting or tracking a protein or cell that has a protein of interest. In the alternatives described herein, the fusion protein provided can comprise a marker sequence that can be selected in experiments, such as flow cytometry. In some embodiments, the marker comprises a truncated Her2 (Her2t) polypeptide, or a truncated EGFR (EGFRt).

As used herein, “suicide gene therapy,” “suicide genes” and “suicide gene systems” have their plain and ordinary meaning when read in light of the specification, and includes, for example, methods to destroy a cell through apoptosis, which requires a suicide gene that will cause a cell to kill itself by apoptosis. Due to safety concerns for the patients in need of using genetically modified immune cells for treatment or modification of a tumor environment, strategies are being developed in order to prevent or abate adverse events. Adverse effects of incorporation of genetically modified immune cells into a subject for a pretreatment step can include “cytokine storms,” which is a cytokine release syndrome, wherein the infused T-cells release cytokines into the bloodstream, which can lead to dangerously high fevers, as well as, a precipitous drop in blood pressure. Control of the system by tamoxifen, as previously described, may also be used when there is indication of a cytokine storm, such as a fever.

As used herein, “vector” or “construct” has its plain and ordinary meaning when read in light of the specification, and includes, for example, a nucleic acid used to introduce heterologous nucleic acids into a cell that has regulatory elements to provide expression of the heterologous nucleic acids in the cell. Vectors include but are not limited to plasmid, minicircles, yeast, viral genomes, lentiviral vector, foamy viral vector, retroviral vector or gammaretroviral vector. The vector may be DNA or RNA, such as mRNA.

As used herein, “T-cells” or “T lymphocytes” can be from any mammal, preferably a primate, including monkeys or humans, a companion animal such as a dog, cat, or horse, or a domestic animal, such as a sheep, goat, or cattle. In some alternatives the T-cells are allogeneic (from the same species but different donor) as the recipient subject; in some alternatives the T-cells are autologous (the donor and the recipient are the same); in some alternatives the T-cells are syngeneic (the donor and the recipients are different but are identical twins).

As used herein, “T cell precursors” refers to lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8 and are therefore classed as double-negative (CD4−CD8−) cells. As they progress through their development, they become double-positive thymocytes (CD4+CD8+), and finally mature to single-positive (CD4+CD8− or CD4−CD8+) thymocytes that are then released from the thymus to peripheral tissues.

As used herein, “hematopoietic stem cells” or “HSC” are precursor cells that can give rise to myeloid cells such as, for example, macrophages, monocytes, macrophages, neutrophils, basophils, eosinophils, erythrocytes, megakaryocytes/platelets, dendritic cells and/or lymphoid lineages (such as, for example, T-cells, B-cells, or NK-cells). HSCs have a heterogeneous population in which three classes of stem cells exist, which are distinguished by their ratio of lymphoid to myeloid progeny in the blood (LM).

As used herein, “CD4+ expressing T-cell,” or “CD4+ T-cell,” are used synonymously throughout, is also known as T helper cells, which play an important role in the immune system, and in the adaptive immune system. CD4+ T-cells also help the activity of other immune cells by releasing T-cell cytokines. These cells help, suppress or regulate immune responses. They are essential in B cell antibody class switching, in the activation and growth of cytotoxic T-cells, and in maximizing bactericidal activity of phagocytes, such as macrophages. CD4+ expressing T-cells have the ability to make some cytokines, however the amounts of cytokines made by CD4+ T-cells are not at a concentration that promotes, improves, contributes to, or induces engraftment fitness. As described herein, “CD4+ T-cells” are mature T helper-cells that play a role in the adaptive immune system.

As used herein, “CD8+ expressing T-cell” or “CD8+ T-cell,” are used synonymously throughout, is also known as a TC, cytotoxic T lymphocyte, CTL, T-killer cell, cytolytic T-cell or killer T-cell. As described herein, CD8+ T-cells are T-lymphocytes that can kill cancer cells, virally infected cells, or damaged cells. CD8+ T-cells express T-cell receptors (TCRs) that can recognize a specific antigen. CD8+ T-cells express CD8 on the surface. CD8+ expressing T-cells have the ability to make some cytokines, however the amounts of cytokines made by CD8+ T-cells are not at a concentration that promotes, improves, contributes to, or induces engraftment fitness. “CD8 T-cells” or “killer T-cells” are T-lymphocytes that can kill cancer cells, cells that are infected with viruses or cells that are damaged.

Mature T cells express the surface protein CD4 and are referred to as CD4+ T-cells. CD4+ T-cells are generally treated as having a pre-defined role as helper T-cells within the immune system. For example, when an antigen-presenting cell expresses an antigen on MHC class 11, a CD4+ cell will aid those cells through a combination of cell to cell interactions (e.g. CD40 and CD40L) and through cytokines. Nevertheless, there are rare exceptions; for example, sub-groups of regulatory T-cells, natural killer cells, and cytotoxic T-cells express CD4. All of the latter CD4+ expressing T-cell groups are not considered T helper cells.

As used herein, “central memory” T-cell (or “TCM”) refers to an antigen experienced CTL that expresses CD62L or CCR-7 and CD45RO on the surface thereof and does not express or has decreased expression of CD45RA as compared to naïve cells. In some embodiments, central memory cells are positive for expression of CD62L, CCR7, CD28, CD127, CD45RO, and/or CD95, and have decreased expression of CD54RA, as compared to naïve cells.

As used herein, “effector memory” T-cell (or “TEM”) refers to an antigen experienced T-cell that does not express or has decreased expression of CD62L on the surface thereof as compared to central memory cells, and does not express or has decreased expression of CD45RA as compared to naïve cell. In some embodiments, effector memory cells are negative for expression of CD62L and/or CCR7, as compared to naïve cells or central memory cells, and have variable expression of CD28 and/or CD45RA.

As used herein, “naïve” T-cells refers to a non-antigen experienced T lymphocyte that expresses CD62L and/or CD45RA, and/or does not express CD45RO− as compared to central or effector memory cells. In some embodiments, naïve CD8+ T lymphocytes are characterized by the expression of phenotypic markers of naïve T-cells including CD62L, CCR7, CD28, CD127, or CD45RA.

As used herein, “effector” “TE” T-cells refers to a antigen experienced cytotoxic T lymphocyte cells that do not express or have decreased expression of CD62L, CCR7, CD28, and are positive for granzyme B or perforin or both, as compared to central memory or naïve T-cells.

As used herein, “protein” has its plain and ordinary meaning when read in light of the specification, and includes, for example, a macromolecule comprising one or more polypeptide chains. A protein can therefore comprise of peptides, which are chains of amino acid monomers linked by peptide (amide) bonds, formed by any one or more of the amino acids. A protein or peptide can contain at least two amino acids, and no limitation is placed on the maximum number of amino acids that can comprise the protein or peptide sequence. Without being limiting, the amino acids are, for example, arginine, histidine, lysine, aspartic acid, glutamic acid, serine, threonine, asparagine, glutamine, cysteine, cystine, glycine, proline, alanine, valine, hydroxyproline, isoleucine, leucine, pyrolysine, methionine, phenylalanine, tyrosine, tryptophan, ornithine, S-adenosylmethionine, or selenocysteine. A protein can also comprise non-peptide components, such as carbohydrate groups, for example. Carbohydrates and other non-peptide substituents can be added to a protein by the cell in which the protein is produced and will vary with the type of cell. Proteins are defined herein in terms of their amino acid backbone structures; substituents such as carbohydrate groups are generally not specified, but can be present nonetheless.

As used herein, “propagating cells” or propagation refers to steps to allow proliferation, expansion, growth and reproduction of cells. For example, cultures of CD8+ T-cells and CD4+ T-cells can typically be incubated under conditions that are suitable for the growth and proliferation of T lymphocytes. In some alternatives of the method of making genetically modified T-cells, which have a chimeric antigen receptor, the CD4+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods. In some alternatives of the method of making genetically modified T-cells, which have a chimeric antigen receptor, the CD8+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods.

In another alternative, the expansion method or propagation can further comprise adding anti-CD3 and/or anti CD28 antibody to the culture medium (e.g., at a concentration of at least 0.5 ng/ml). In another alternative, the method of making genetically modified T-cells, which have a chimeric antigen receptor method can further comprise adding IL-2, IL-15, or IL-21 or any combination thereof to the culture medium (e.g., wherein the concentration of IL-2 is at least 10 units/ml). In another alternative, the method of making genetically modified T-cells, which have a chimeric antigen receptor method can further comprise adding IL-7, IL-5, or IL21 or any combination thereof to the culture medium (e.g., wherein the concentration of IL-2 is at least 10 units/ml). After isolation of T lymphocytes, both cytotoxic and helper T lymphocytes can be sorted into naïve, memory, and effector T-cell subpopulations either before or after expansion.

“Subject” or “patient,” as described herein, refers to any organism upon which the embodiments described herein may be used or administered, e.g., for experimental, diagnostic, prophylactic, and/or therapeutic purposes. Subjects or patients include, for example, animals. In some embodiments, the subject is mice, rats, rabbits, non-human primates, or humans. In some embodiments, the subject is a cow, sheep, pig, horse, dog, cat, primate or a human.

“Cancer,” as described herein, is a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, leukemia, multiple myeloma, or brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, or prostate cancer. Examples include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia.

In some embodiments, a method of treating, ameliorating, or inhibiting one or more of the aforementioned cancers in a subject is provided, wherein such methods are practiced by administering any one of the cells described herein to a subject in need thereof, wherein the cell comprises a chimeric antigen receptor (CAR); optionally wherein said subject is selected or identified to receive a medicament for said disorder, such as by clinical or diagnostic evaluation for the presence of said disorder. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer. In some embodiments, the subject that receives one of the therapies set forth herein and is also selected to receive an additional cancer therapy, which can include a cancer therapeutic, radiation, chemotherapy, or a cancer therapy drug. In some embodiments, the cancer therapy drug provided comprises Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

Some embodiments include polypeptide sequences or conservative variations thereof, such as conservative substitutions in a polypeptide sequence. In some embodiments, “conservative amino acid substitution” refers to amino acid substitutions that substitute functionally equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in TABLE 1.

TABLE 1

| Family | Amino Acids |
|---|---|
| non-polar | Trp, Phe, Met, Leu, Ile, Val, Ala, Pro |
| uncharged polar | Gly, Ser, Thr, Asn, Gln, Tyr, Cys |
| acidic/negatively charged | Asp, Glu |
| basic/positively charged | Arg, Lys, His |
| Beta-branched | Thr, Val, Ile |
| residues that influence chain | Gly, Pro |
| orientation | |
| aromatic | Trp, Tyr, Phe, His |

### Certain Polypeptides

Some embodiments of the methods and compositions provided herein include a polynucleotide encoding a chimeric polypeptide, such as a PD1 chimeric polypeptide comprising a programmed cell death protein 1 (PD1) extracellular domain, and an intracellular immunostimulatory domain. In some embodiments, the polynucleotide comprises a first nucleic acid encoding the PD1 domain; and a second nucleic acid encoding the immunostimulatory domain.

In some embodiments, the PD1 extracellular domain comprises a wild type PD1 extracellular domain. In some embodiments, the PD1 extracellular domain comprises an A99L substitution. In some embodiments, the PD1 extracellular domain comprises a polypeptide encoded by a nucleotide sequence having a percentage sequence identity to the nucleotide sequence of SEQ ID NO:01 or SEQ ID NO:02 of at least 90%, 91%, 92%, 93%, 94%, 95%, 96%, 97%, 98%, or 99% or within a range defined by any two of the aforementioned percentages.

In some embodiments, the intracellular immunostimulatory domain is an intracellular domain of a protein selected from myeloid differentiation primary response 88 protein (MYD88), CD28, or CD2. In some embodiments, the intracellular immunostimulatory domain comprises MYD88 or CD2. In some embodiments, the intracellular immunostimulatory domain comprises a polypeptide encoded by a nucleotide sequence having a percentage sequence identity to the nucleotide sequence of any one of SEQ ID NOs:03-05 of at least 90%, 91%, 92%, 93%, 94%, 95%, 96%, 97%, 98%, or 99% or within a range defined by any two of the aforementioned percentages.

In some embodiments, the PD1 domain is linked to the intracellular immunostimulatory domain via a transmembrane domain. In some embodiments, the transmembrane domain comprises a PD1 transmembrane domain. In some embodiments, the PD1 transmembrane domain is encoded by the nucleotide sequence of SEQ ID NO:12.

In some embodiments, the transmembrane domain is linked to the intracellular immunostimulatory domain via a linker. In some embodiments, the linker comprises a glycine linker, such as a glycine linker comprising or consisting of 2, 3, 4, 5, 6, 7, 8, 9, or 10 consecutive glycine residues. In some embodiments, the linker is encoded by the nucleotide sequence of SEQ ID NO:13.

In some embodiments, the PD1 chimeric polypeptide is encoded by a nucleotide sequence having a percentage sequence identity to the nucleotide sequence of any one of SEQ ID NOs:06-12 of at least 90%, 91%, 92%, 93%, 94%, 95%, 96%, 97%, 98%, or 99% or within a range defined by any two of the aforementioned percentages.

In some embodiments, the polynucleotide also includes a third nucleic acid encoding a cell surface selectable marker. In some embodiments, the cell surface selectable marker is selected from a truncated HER2 (Her2tG) polypeptide, a truncated EGFR (EGFRt) polypeptide, or a truncated CD19 (CD19t). In some embodiments, the second nucleic acid and the third nucleic acid are linked via a ribosomal skip sequence. In some embodiments, the ribosomal skip sequence is selected from P2A, T2A, E2A or F2A.

In some embodiments, the polynucleotide also includes a constitutive promoter operably linked to the first nucleic acid. In some embodiments, the constitutive promoter comprises an EF1α promoter. In some embodiments, the polynucleotide also includes an inducible promoter operably linked to the first nucleic acid. Examples of inducible promoters include inducible synthetic promoter (iSynPro) sequences disclosed in U.S. 2020/0095573, which is hereby expressly incorporated by reference in its entirety.

In some embodiments, the polynucleotide also includes an inducible cytotoxic gene. In some embodiments, the cytotoxic gene encodes a protein selected from a thymidine kinase, thymidine kinase fused to thymidylate kinase, oxidoreductase, deoxycytidine kinase, uracil phosphoribosyltransferase, cytosine deaminase, or cytosine deaminase fused to uracil phosphoribosyltransferase. In some embodiments, the cytotoxic gene encodes a thymidine kinase.

Some embodiments of the methods and compositions provided herein include a vector comprising any one of the polynucleotides provided herein. In some embodiments, the vector comprises a viral vector. In some embodiments, the vector is selected from a lentiviral vector, an adeno-associated viral vector, or an adenoviral vector. In some embodiments, the vector comprises a lentiviral vector.

Some embodiments of the methods and compositions provided herein include a polypeptide, such as PD1 chimeric polypeptide, encoded by any one of the polynucleotides provided herein. TABLE 2 lists certain amino acid and nucleotide sequences useful with certain embodiments.

TABLE 2

| Feature | |
|---|---|
| [SEQ ID | |
| NO] | Sequence |
| PD1 wild- | ATGCAGATCCCTCAGGCCCCTTGGCCTGTC |
| type domain | GTGTGGGCTGTGCTGCAGCTGGGATGGCGG |
| [SEQ ID | CCTGGCTGGTTTCTGGACAGCCCCGACAGA |
| NO: 01] | CCCTGGAACCCCCCTACATTTTCCCCTGCC |
| | CTGCTGGTCGTGACCGAGGGCGACAATGCC |
| | ACCTTCACCTGTAGCTTCAGCAACACCAGC |
| | GAGAGCTTCGTGCTGAACTGGTACAGAATG |
| | AGCCCCAGCAACCAGACCGACAAGCTGGCC |
| | GCCTTCCCCGAGGATAGATCTCAGCCCGGC |
| | CAGGACTGCCGGTTCAGAGTGACCCAGCTG |
| | CCCAACGGCCGGGACTTCCACATGTCTGTC |
| | GTGCGGGCCAGACGGAACGACAGCGGCACA |
| | TATCTGTGCGGCGCCATCAGCCTGGCCCCC |
| | AAGGCCCAGATCAAAGAGAGCCTGAGAGCC |
| | GAGCTGAGAGTGACCGAGAGAAGGGCCGAA |
| | GTGCCTACCGCCCACCCTAGCCCATCTCCA |
| | AGACCTGCCGGCCAGTTCCAGACACTGGTC |
| | GTGGGAGTCGTGGGCGGACTGCTGGGATCT |
| | CTGGTGCTGCTCGTGTGGGTGCTGGCCGTG |
| | ATT |
| PD1 (A99L) | ATGCAGATCCCACAGGCGCCCTGGCCAGTC |
| domain | GTCTGGGCGGTGCTACAACTGGGCTGGCGG |
| [SEQ ID | CCAGGATGGTTCTTAGACTCCCCAGACAGG |
| NO: 02] | CCCTGGAACCCCCCCACCTTCTCCCCAGCC |
| | CTGCTCGTGGTGACCGAAGGGGACAACGCC |
| | ACCTTCACCTGCAGCTTCTCCAACACATCG |
| | GAGAGCTTCGTGCTAAACTGGTACCGCATG |
| | AGCCCCAGCAACCAGACGGACAAGCTGGCC |
| | GCCTTCCCCGAGGACCGCAGCCAGCCCGGC |
| | CAGGACTGCCGCTTCCGTGTCACACAACTG |
| | CCCAACGGGCGTGACTTCCACATGAGCGTG |
| | GTCAGGGCCCGGCGCAATGACAGCGGCACC |
| | TACCTCTGTGGGGCCATCTCCCTGGCCCCC |
| | AAGCTCCAGATCAAAGAGAGCCTGCGGGCA |
| | GAGCTCAGGGTGACAGAGAGAAGGGCAGAA |
| | GTGCCCACAGCCCAC |
| CD28 | TGTCCAAGTCCCCTATTTCCCGGACCTTCT |
| intracellular | AAGCCCTTTTGGGTGCTGGTGGTGGTTGGT |
| domain | GGAGTCCTGGCTTGCTATAGCTTGCTAGTA |
| [SEQ ID | ACAGTGGCCTTTATTATTTTCTGGGTGAGG |
| NO: 03] | AGTAAGAGGAGCAGGCTCCTGCACAGTGAC |
| | TACATGAACATGACTCCCCGCCGCCCCGGG |
| | CCCACCCGCAAGCATTACCAGCCCTATGCC |
| | CCACCACGCGACTTCGCAGCCTATCGCTCC |
| MyD88 | ATGGCTGCTGGCGGACCTGGCGCTGGATCT |
| domain | GCTGCTCCTGTGTCTAGCACCAGCAGCCTG |
| [SEQ ID | CCTCTGGCCGCCCTGAATATGAGAGTGCGG |
| NO: 04] | CGGAGACTGAGCCTGTTCCTGAACGTGCGG |
| | ACACAGGTGGCCGCCGATTGGACAGCTCTG |
| | GCCGAGGAAATGGACTTCGAGTACCTGGAA |
| | ATCCGGCAGCTGGAAACCCAGGCCGACCCT |
| | ACAGGACGCCTGCTGGATGCTTGGCAGGGC |
| | AGACCAGGCGCTTCTGTGGGGAGACTGCTG |
| | GAACTGCTGACCAAGCTGGGCCGGGACGAC |
| | GTGCTGCTGGAACTGGGCCCTAGCATCGAA |
| | GAGGACTGCCAGAAGTACATCCTGAAGCAG |
| | CAGCAGGAAGAGGCCGAGAAGCCTCTGCAG |
| | GTGGCAGCCGTGGATAGCAGCGTGCCAAGA |
| | ACAGCTGAGCTGGCCGGAATCACCACCCTG |
| | GACGATCCTCTGGGCCACATGCCCGAGAGA |
| | TTCGACGCCTTCATCTGCTACTGCCCCAGC |
| | GACATCCAGTTCGTGCAGGAAATGATCAGA |
| | CAGCTGGAACAGACCAACTACCGGCTGAAG |
| | CTGTGCGTGTCCGACCGGGATGTGCTGCCT |
| | GGCACCTGTGTGTGGTCTATCGCCAGCGAG |
| | CTGATCGAGAAGCGGTGCAGACGGATGGTC |
| | GTGGTGGTGTCCGACGACTACCTGCAGTCC |
| | AAAGAGTGCGACTTCCAGACCAAGTTCGCC |
| | CTGAGCCTGAGCCCTGGCGCCCACCAGAAG |
| | AGACTGATCCCCATCAAGTACAAGGCCATG |
| | AAGAAAGAGTTCCCCAGCATCCTGCGGTTC |
| | ATCACCGTGTGCGACTACACCAACCCCTGC |
| | ACCAAGTCCTGGTTCTGGACCAGACTGGCC |
| | AAGGCCCTGTCTCTGCCT |
| CD2 domain | AAGCGGAAGAAGCAGCGGAGCAGAAGAAAC |
| [SEQ ID | GACGAGGAACTGGAAACCAGAGCCCACAGA |
| NO: 05] | GTGGCCACCGAGGAAAGAGGCAGAAAGCCC |
| | CACCAGATCCCCGCCTCCACCCCTCAGAAT |
| | CCTGCCACCTCTCAGCACCCTCCACCTCCC |
| | CCAGGACACAGAAGCCAGGCCCCTAGCCAT |
| | AGACCTCCACCACCCGGACACCGGGTGCAG |
| | CATCAGCCTCAGAAAAGACCCCCTGCCCCT |
| | AGCGGCACACAGGTGCACCAGCAGAAAGGC |
| | CCCCCACTGCCCAGACCTAGAGTGCAGCCT |
| | AAGCCTCCTCACGGCGCTGCCGAGAATAGC |
| | CTGAGCCCCTCCAGCAAC |
| PD1(WT): | ATGCAGATCCCACAGGCGCCCTGGCCAGTC |
| CD28 | GTCTGGGCGGTGCTACAACTGGGCTGGCGG |
| [SEQ ID | CCAGGATGGTTCTTAGACTCCCCAGACAGG |
| NO: 06] | CCCTGGAACCCCCCCACCTTCTCCCCAGCC |
| | CTGCTCGTGGTGACCGAAGGGGACAACGCC |
| | ACCTTCACCTGCAGCTTCTCCAACACATCG |
| | GAGAGCTTCGTGCTAAACTGGTACCGCATG |
| | AGCCCCAGCAACCAGACGGACAAGCTGGCC |
| | GCCTTCCCCGAGGACCGCAGCCAGCCCGGC |
| | CAGGACTGCCGCTTCCGTGTCACACAACTG |
| | CCCAACGGGCGTGACTTCCACATGAGCGTG |
| | GTCAGGGCCCGGCGCAATGACAGCGGCACC |
| | TACCTCTGTGGGGCCATCTCCCTGGCCCCC |
| | AAGGCGCAGATCAAAGAGAGCCTGCGGGCA |
| | GAGCTCAGGGTGACAGAGAGAAGGGCAGAA |
| | GTGCCCACAGCCCACTGTCCAAGTCCCCTA |
| | TTTCCCGGACCTTCTAAGCCCTTTTGGGTG |
| | CTGGTGGTGGTTGGTGGAGTCCTGGCTTGC |
| | TATAGCTTGCTAGTAACAGTGGCCTTTATT |
| | ATTTTCTGGGTGAGGAGTAAGAGGAGCAGG |
| | CTCCTGCACAGTGACTACATGAACATGACT |
| | CCCCGCCGCCCCGGGCCCACCCGCAAGCAT |
| | TACCAGCCCTATGCCCCACCACGCGACTTC |
| | GCAGCCTATCGCTCC |
| PD1(WT): | ATGCAGATCCCTCAGGCCCCTTGGCCTGTC |
| MYD88 | GTGTGGGCTGTGCTGCAGCTGGGATGGCGG |
| [SEQ ID | CCTGGCTGGTTTCTGGACAGCCCCGACAGA |
| NO: 07] | CCCTGGAACCCCCCTACATTTTCCCCTGCC |
| | CTGCTGGTCGTGACCGAGGGCGACAATGCC |
| | ACCTTCACCTGTAGCTTCAGCAACACCAGC |
| | GAGAGCTTCGTGCTGAACTGGTACAGAATG |
| | AGCCCCAGCAACCAGACCGACAAGCTGGCC |
| | GCCTTCCCCGAGGATAGATCTCAGCCCGGC |
| | CAGGACTGCCGGTTCAGAGTGACCCAGCTG |
| | CCCAACGGCCGGGACTTCCACATGTCTGTC |
| | GTGCGGGCCAGACGGAACGACAGCGGCACA |
| | TATCTGTGCGGCGCCATCAGCCTGGCCCCC |
| | AAGGCCCAGATCAAAGAGAGCCTGAGAGCC |
| | GAGCTGAGAGTGACCGAGAGAAGGGCCGAA |
| | GTGCCTACCGCCCACCCTAGCCCATCTCCA |
| | AGACCTGCCGGCCAGTTCCAGACACTGGTC |
| | GTGGGAGTCGTGGGCGGACTGCTGGGATCT |
| | CTGGTGCTGCTCGTGTGGGTGCTGGCCGTG |
| | ATTGGCGGAGGAATGGCTGCTGGCGGACCT |
| | GGCGCTGGATCTGCTGCTCCTGTGTCTAGC |
| | ACCAGCAGCCTGCCTCTGGCCGCCCTGAAT |
| | ATGAGAGTGCGGCGGAGACTGAGCCTGTTC |
| | CTGAACGTGCGGACACAGGTGGCCGCCGAT |
| | TGGACAGCTCTGGCCGAGGAAATGGACTTC |
| | GAGTACCTGGAAATCCGGCAGCTGGAAACC |
| | CAGGCCGACCCTACAGGACGCCTGCTGGAT |
| | GCTTGGCAGGGCAGACCAGGCGCTTCTGTG |
| | GGGAGACTGCTGGAACTGCTGACCAAGCTG |
| | GGCCGGGACGACGTGCTGCTGGAACTGGGC |
| | CCTAGCATCGAAGAGGACTGCCAGAAGTAC |
| | ATCCTGAAGCAGCAGCAGGAAGAGGCCGAG |
| | AAGCCTCTGCAGGTGGCAGCCGTGGATAGC |
| | AGCGTGCCAAGAACAGCTGAGCTGGCCGGA |
| | ATCACCACCCTGGACGATCCTCTGGGCCAC |
| | ATGCCCGAGAGATTCGACGCCTTCATCTGC |
| | TACTGCCCCAGCGACATCCAGTTCGTGCAG |
| | GAAATGATCAGACAGCTGGAACAGACCAAC |
| | TACCGGCTGAAGCTGTGCGTGTCCGACCGG |
| | GATGTGCTGCCTGGCACCTGTGTGTGGTCT |
| | ATCGCCAGCGAGCTGATCGAGAAGCGGTGC |
| | AGACGGATGGTCGTGGTGGTGTCCGACGAC |
| | TACCTGCAGTCCAAAGAGTGCGACTTCCAG |
| | ACCAAGTTCGCCCTGAGCCTGAGCCCTGGC |
| | GCCCACCAGAAGAGACTGATCCCCATCAAG |
| | TACAAGGCCATGAAGAAAGAGTTCCCCAGC |
| | ATCCTGCGGTTCATCACCGTGTGCGACTAC |
| | ACCAACCCCTGCACCAAGTCCTGGTTCTGG |
| | ACCAGACTGGCCAAGGCCCTGTCTCTGCCT |
| PD1(WT): | ATGCAGATCCCTCAGGCCCCTTGGCCTGTC |
| CD2 | GTGTGGGCTGTGCTGCAGCTGGGATGGCGG |
| [SEQ ID | CCTGGCTGGTTTCTGGACAGCCCCGACAGA |
| NO: 08] | CCCTGGAACCCCCCTACATTTTCCCCTGCC |
| | CTGCTGGTCGTGACCGAGGGCGACAATGCC |
| | ACCTTCACCTGTAGCTTCAGCAACACCAGC |
| | GAGAGCTTCGTGCTGAACTGGTACAGAATG |
| | AGCCCCAGCAACCAGACCGACAAGCTGGCC |
| | GCCTTCCCCGAGGATAGATCTCAGCCCGGC |
| | CAGGACTGCCGGTTCAGAGTGACCCAGCTG |
| | CCCAACGGCCGGGACTTCCACATGTCTGTC |
| | GTGCGGGCCAGACGGAACGACAGCGGCACA |
| | TATCTGTGCGGCGCCATCAGCCTGGCCCCC |
| | AAGGCCCAGATCAAAGAGAGCCTGAGAGCC |
| | GAGCTGAGAGTGACCGAGAGAAGGGCCGAA |
| | GTGCCTACCGCCCACCCTAGCCCATCTCCA |
| | AGACCTGCCGGCCAGTTCCAGACACTGGTC |
| | GTGGGAGTCGTGGGCGGACTGCTGGGATCT |
| | CTGGTGCTGCTCGTGTGGGTGCTGGCCGTG |
| | ATTGGCGGAGGAAAGCGGAAGAAGCAGCGG |
| | AGCAGAAGAAACGACGAGGAACTGGAAACC |
| | AGAGCCCACAGAGTGGCCACCGAGGAAAGA |
| | GGCAGAAAGCCCCACCAGATCCCCGCCTCC |
| | ACCCCTCAGAATCCTGCCACCTCTCAGCAC |
| | CCTCCACCTCCCCCAGGACACAGAAGCCAG |
| | GCCCCTAGCCATAGACCTCCACCACCCGGA |
| | CACCGGGTGCAGCATCAGCCTCAGAAAAGA |
| | CCCCCTGCCCCTAGCGGCACACAGGTGCAC |
| | CAGCAGAAAGGCCCCCCACTGCCCAGACCT |
| | AGAGTGCAGCCTAAGCCTCCTCACGGCGCT |
| | GCCGAGAATAGCCTGAGCCCCTCCAGCAAC |
| PD1(A99L): | ATGCAGATCCCACAGGCGCCCTGGCCAGTC |
| CD28 | GTCTGGGCGGTGCTACAACTGGGCTGGCGG |
| [SEQ ID | CCAGGATGGTTCTTAGACTCCCCAGACAGG |
| NO: 09] | CCCTGGAACCCCCCCACCTTCTCCCCAGCC |
| | CTGCTCGTGGTGACCGAAGGGGACAACGCC |
| | ACCTTCACCTGCAGCTTCTCCAACACATCG |
| | GAGAGCTTCGTGCTAAACTGGTACCGCATG |
| | AGCCCCAGCAACCAGACGGACAAGCTGGCC |
| | GCCTTCCCCGAGGACCGCAGCCAGCCCGGC |
| | CAGGACTGCCGCTTCCGTGTCACACAACTG |
| | CCCAACGGGCGTGACTTCCACATGAGCGTG |
| | GTCAGGGCCCGGCGCAATGACAGCGGCACC |
| | TACCTCTGTGGGGCCATCTCCCTGGCCCCC |
| | AAGCTCCAGATCAAAGAGAGCCTGCGGGCA |
| | GAGCTCAGGGTGACAGAGAGAAGGGCAGAA |
| | GTGCCCACAGCCCACTGTCCAAGTCCCCTA |
| | TTTCCCGGACCTTCTAAGCCCTTTTGGGTG |
| | CTGGTGGTGGTTGGTGGAGTCCTGGCTTGC |
| | TATAGCTTGCTAGTAACAGTGGCCTTTATT |
| | ATTTTCTGGGTGAGGAGTAAGAGGAGCAGG |
| | CTCCTGCACAGTGACTACATGAACATGACT |
| | CCCCGCCGCCCCGGGCCCACCCGCAAGCAT |
| | TACCAGCCCTATGCCCCACCACGCGACTTC |
| | GCAGCCTATCGCTCC |
| PD1(A99L): | ATGCAGATCCCTCAGGCCCCTTGGCCTGTC |
| MYD88 | GTGTGGGCTGTGCTGCAGCTGGGATGGCGG |
| [SEQ ID | CCTGGCTGGTTTCTGGACAGCCCCGACAGA |
| NO: 10] | CCCTGGAACCCCCCTACATTTTCCCCTGCC |
| | CTGCTGGTCGTGACCGAGGGCGACAATGCC |
| | ACCTTCACCTGTAGCTTCAGCAACACCAGC |
| | GAGAGCTTCGTGCTGAACTGGTACAGAATG |
| | AGCCCCAGCAACCAGACCGACAAGCTGGCC |
| | GCCTTCCCCGAGGATAGATCTCAGCCCGGC |
| | CAGGACTGCCGGTTCAGAGTGACCCAGCTG |
| | CCCAACGGCCGGGACTTCCACATGTCTGTC |
| | GTGCGGGCCAGACGGAACGACAGCGGCACA |
| | TATCTGTGCGGCGCCATCAGCCTGGCCCCC |
| | AAGCTCCAGATCAAAGAGAGCCTGAGAGCC |
| | GAGCTGAGAGTGACCGAGAGAAGGGCCGAA |
| | GTGCCTACCGCCCACCCTAGCCCATCTCCA |
| | AGACCTGCCGGCCAGTTCCAGACACTGGTC |
| | GTGGGAGTCGTGGGCGGACTGCTGGGATCT |
| | CTGGTGCTGCTCGTGTGGGTGCTGGCCGTG |
| | ATTGGCGGAGGAATGGCTGCTGGCGGACCT |
| | GGCGCTGGATCTGCTGCTCCTGTGTCTAGC |
| | ACCAGCAGCCTGCCTCTGGCCGCCCTGAAT |
| | ATGAGAGTGCGGCGGAGACTGAGCCTGTTC |
| | CTGAACGTGCGGACACAGGTGGCCGCCGAT |
| | TGGACAGCTCTGGCCGAGGAAATGGACTTC |
| | GAGTACCTGGAAATCCGGCAGCTGGAAACC |
| | CAGGCCGACCCTACAGGACGCCTGCTGGAT |
| | GCTTGGCAGGGCAGACCAGGCGCTTCTGTG |
| | GGGAGACTGCTGGAACTGCTGACCAAGCTG |
| | GGCCGGGACGACGTGCTGCTGGAACTGGGC |
| | CCTAGCATCGAAGAGGACTGCCAGAAGTAC |
| | ATCCTGAAGCAGCAGCAGGAAGAGGCCGAG |
| | AAGCCTCTGCAGGTGGCAGCCGTGGATAGC |
| | AGCGTGCCAAGAACAGCTGAGCTGGCCGGA |
| | ATCACCACCCTGGACGATCCTCTGGGCCAC |
| | ATGCCCGAGAGATTCGACGCCTTCATCTGC |
| | TACTGCCCCAGCGACATCCAGTTCGTGCAG |
| | GAAATGATCAGACAGCTGGAACAGACCAAC |
| | TACCGGCTGAAGCTGTGCGTGTCCGACCGG |
| | GATGTGCTGCCTGGCACCTGTGTGTGGTCT |
| | ATCGCCAGCGAGCTGATCGAGAAGCGGTGC |
| | AGACGGATGGTCGTGGTGGTGTCCGACGAC |
| | TACCTGCAGTCCAAAGAGTGCGACTTCCAG |
| | ACCAAGTTCGCCCTGAGCCTGAGCCCTGGC |
| | GCCCACCAGAAGAGACTGATCCCCATCAAG |
| | TACAAGGCCATGAAGAAAGAGTTCCCCAGC |
| | ATCCTGCGGTTCATCACCGTGTGCGACTAC |
| | ACCAACCCCTGCACCAAGTCCTGGTTCTGG |
| | ACCAGACTGGCCAAGGCCCTGTCTCTGCCT |
| PD1(A99L): | ATGCAGATCCCTCAGGCCCCTTGGCCTGTC |
| CD2 | GTGTGGGCTGTGCTGCAGCTGGGATGGCGG |
| [SEQ ID | CCTGGCTGGTTTCTGGACAGCCCCGACAGA |
| NO: 11] | CCCTGGAACCCCCCTACATTTTCCCCTGCC |
| | CTGCTGGTCGTGACCGAGGGCGACAATGCC |
| | ACCTTCACCTGTAGCTTCAGCAACACCAGC |
| | GAGAGCTTCGTGCTGAACTGGTACAGAATG |
| | AGCCCCAGCAACCAGACCGACAAGCTGGCC |
| | GCCTTCCCCGAGGATAGATCTCAGCCCGGC |
| | CAGGACTGCCGGTTCAGAGTGACCCAGCTG |
| | CCCAACGGCCGGGACTTCCACATGTCTGTC |
| | GTGCGGGCCAGACGGAACGACAGCGGCACA |
| | TATCTGTGCGGCGCCATCAGCCTGGCCCCC |
| | AAGCTCCAGATCAAAGAGAGCCTGAGAGCC |
| | GAGCTGAGAGTGACCGAGAGAAGGGCCGAA |
| | GTGCCTACCGCCCACCCTAGCCCATCTCCA |
| | AGACCTGCCGGCCAGTTCCAGACACTGGTC |
| | GTGGGAGTCGTGGGCGGACTGCTGGGATCT |
| | CTGGTGCTGCTCGTGTGGGTGCTGGCCGTG |
| | ATTGGCGGAGGAAAGCGGAAGAAGCAGCGG |
| | AGCAGAAGAAACGACGAGGAACTGGAAACC |
| | AGAGCCCACAGAGTGGCCACCGAGGAAAGA |
| | GGCAGAAAGCCCCACCAGATCCCCGCCTCC |
| | ACCCCTCAGAATCCTGCCACCTCTCAGCAC |
| | CCTCCACCTCCCCCAGGACACAGAAGCCAG |
| | GCCCCTAGCCATAGACCTCCACCACCCGGA |
| | CACCGGGTGCAGCATCAGCCTCAGAAAAGA |
| | CCCCCTGCCCCTAGCGGCACACAGGTGCAC |
| | CAGCAGAAAGGCCCCCCACTGCCCAGACCT |
| | AGAGTGCAGCCTAAGCCTCCTCACGGCGCT |
| | GCCGAGAATAGCCTGAGCCCCTCCAGCAAC |
| PD1tm | GTGGGAGTCGTGGGCGGACTGCTGGGATCT |
| [SEQ ID | CTGGTGCTGCTCGTGTGGGTGCTGGCCGTG |
| NO: 12] | ATT |
| GLY3 linker | GGCGGAGGA |
| [SEQ ID | |
| NO: 13] | |
| Inducible | CTGCTTAGGGTTAGGCGTTTTGCGCTGCTT |
| synthetic | CGCGATCGAATGAGTCACATCGATCTCCGC |
| promoter, | CCCCTCTTCGAGGGGGCGGGGTCGAGGAGG |
| iSynPro | AAAAACTCGAATGAGTCACATCGACCCTTT |
| [SEQ ID | GATCTTCGAGGGGACTTTCCGGGGTGGAGC |
| NO: 14] | AAGCGTGACAAGTCCACGTATGACCCGACC |
| | GACGATATCGAAGCCTACGCGCTGAACGCC |
| | AGCCCCGATCGACCCCGCCCCCTCGATTTC |
| | CAAGAAATCGAATGACATCATCTTTCGAAT |
| | GACATCATCTTTCGAGGGGACTTTCCTCGA |
| | ACTTCCTTCGAGGGGACTTTCCTCGAGGGG |
| | ACTTTCCTCGAGGAGGAAAAACTCGAGTAG |
| | AGTCTAGACTCTACATTTTGACACCCCCA |

### Certain Cells

Some embodiments of the methods and compositions provided herein include a cell or cell population comprising any one of the chimeric polypeptides provided herein, such as a PD1 chimeric polypeptide, or any one of the polynucleotides encoding such chimeric polypeptide.

In some embodiments, the cell also includes a fourth nucleic acid encoding a chimeric antigen receptor (CAR), and/or a CAR protein. In some embodiments, the CAR is capable of or configured to specifically bind to a target antigen expressed by a cancer cell. In some embodiments, the target antigen is expressed by a cell of a solid tumor. In some embodiments, the target antigen comprises CD171.

In some embodiments, the cell is a T cell. In some embodiments, the cell is derived from a CD4+ T cell, a CD8+ T cell, a precursor T cell, or a hematopoietic stem cell. In some embodiments, the CD8+ T cell is a CD8+ cytotoxic T lymphocyte cell selected from the group consisting of a naïve CD8+ T cell, a central memory CD8+ T cell, an effector memory CD8+ T cell, and a bulk CD8+ T cell. In some embodiments, the CD4+ cell is a CD4+ helper T lymphocyte cell selected from the group consisting of a naïve CD4+ T cell, a central memory CD4+ T cell, an effector memory CD4+ T cell, and a bulk CD4+ T cell.

In some embodiments, the cell is a primary cell. In some embodiments, the cell is mammalian. In some embodiments, the cell is human. In some embodiments, the cell is ex vivo. In some embodiments, the cell is allogenic to a subject, preferably a human, and in other embodiments, the cell is autologous to a subject, preferably a human.

Some embodiments of the methods and compositions provided herein include pharmaceutical compositions comprising any one of the cells provided herein and a pharmaceutically acceptable excipient.

### Certain Methods for Preparing Cells

Some embodiments of the methods and compositions provided herein include preparing an effector cell. Some such methods include introducing any one of the polynucleotides or vectors disclosed herein into a cell.

Some embodiments of the methods and compositions provided herein include preparing an effector cell having an enhanced cytotoxic activity. Some such methods include introducing any one of the polynucleotides or vectors disclosed herein into a cell. In some embodiments, the effector cell comprises a chimeric antigen receptor (CAR) capable of specifically binding to a target antigen. In some embodiments, the effector cell has an enhanced cytotoxic activity in the presence of the target antigen compared to a cell lacking the polynucleotide. In some embodiments, the enhanced cytotoxic activity comprises an increased level of cytokine expression, and/or an increased level of cytolysis of a target cell expressing the target antigen. In some embodiments, the cytokine is selected from IL-2, IFN-gamma, TNF-alpha, or granzyme B.

In some embodiments, the target antigen is a cancer antigen. In some embodiments, the target antigen comprises a solid tumor cell surface antigen. In some embodiments, the target antigen comprises CD171, CD19, or an EGFR.

In some embodiments, the effector cell is a natural killer cell. In some embodiments, the effector cell is a T cell. In some embodiments, the effector cell is a CD4+ T cell, a CD8+ T cell, a precursor T cell, or a hematopoietic stem cell. In some embodiments, the CD8+ T cell is a CD8+ cytotoxic T lymphocyte cell selected from the group consisting of a naïve CD8+ T cell, a central memory CD8+ T cell, an effector memory CD8+ T cell, and a bulk CD8+ T cell, and the CD4+ cell is a CD4+ helper T lymphocyte cell selected from the group consisting of a naïve CD4+ T cell, a central memory CD4+ T cell, an effector memory CD4+ T cell, and a bulk CD4+ T cell. In some embodiments, the effector cell is a primary cell. In some embodiments, the effector cell is mammalian. In some embodiments, the effector cell is human. In some embodiments, the effector cell is ex vivo.

### Certain Methods for Inhibiting a Target Cell

Some embodiments of the methods and compositions provided herein include inhibiting a target cell expressing a target antigen. Some such methods include contacting the target cell with an effector cell comprising any one of the polynucleotides or vectors disclosed herein. In some embodiments, the effector cell comprises a chimeric antigen receptor (CAR) capable of specifically binding to the target antigen.

In some embodiments, the target cell is a cancer cell, such as a solid tumor cancer cell. In some embodiments, the target antigen is a cancer antigen. In some embodiments, the target antigen comprises a solid tumor cell surface antigen. In some embodiments, the target antigen comprises CD171, CD19, or an EGFR.

In some embodiments, the effector cell is a natural killer cell. In some embodiments, the effector cell is a T cell. In some embodiments, the effector cell is a CD4+ T cell, a CD8+ T cell, a precursor T cell, or a hematopoietic stem cell. In some embodiments, the CD8+ T cell is a CD8+ cytotoxic T lymphocyte cell selected from the group consisting of a naïve CD8+ T cell, a central memory CD8+ T cell, an effector memory CD8+ T cell, and a bulk CD8+ T cell; and the CD4+ cell is a CD4+ helper T lymphocyte cell selected from the group consisting of a naïve CD4+ T cell, a central memory CD4+ T cell, an effector memory CD4+ T cell, and a bulk CD4+ T cell. In some embodiments, the effector cell is a primary cell. In some embodiments, the effector cell is mammalian. In some embodiments, the effector cell is human. In some embodiments, the effector cell is ex vivo.

### Certain Therapeutic Methods

Some embodiments of the methods and compositions provided herein relate to therapies. Some embodiments of the methods and compositions provided herein relate to methods of treating, ameliorating or inhibiting a disorder in a subject, comprising administering any one of the cells provided herein to the subject in need thereof. In some embodiments, the cell comprises a chimeric polypeptide, such as a PD1 chimeric polypeptide, and a CAR In some embodiments, the disorder comprises a cancer comprising a target antigen, wherein the CAR is capable of specifically binding to the target antigen. For example, a target antigen can include an antigen expressed at higher levels by a cancer cell than by a non-cancer cell, or an antigen absent from a non-cancer cell. In some embodiments, the cancer is selected from a solid tumor such as a colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma. In some embodiments, the cell is allogenic to the subject. In some embodiments, the cell is autologous to a subject. In some embodiments, the cell is not autologous to the subject that receives the cell. In some embodiments, the subject is mammalian. In some embodiments, the subject is human. Some embodiments of the methods and compositions provided herein relate to any one of the cells or cell populations provided herein for use in treating, ameliorating or inhibiting a disorder such as a cancer in a subject, preferably a subject selected or identified to receive any one or more of the cells or cell populations described herein, e.g., such subjects can be selected or identified on the basis of clinical and/or diagnostic evaluation for the particular disease, such as a cancer, for which the cells or cell populations are being administered.

### EXAMPLES

### Example 1—Construction of PD1 Chimeric Polypeptides

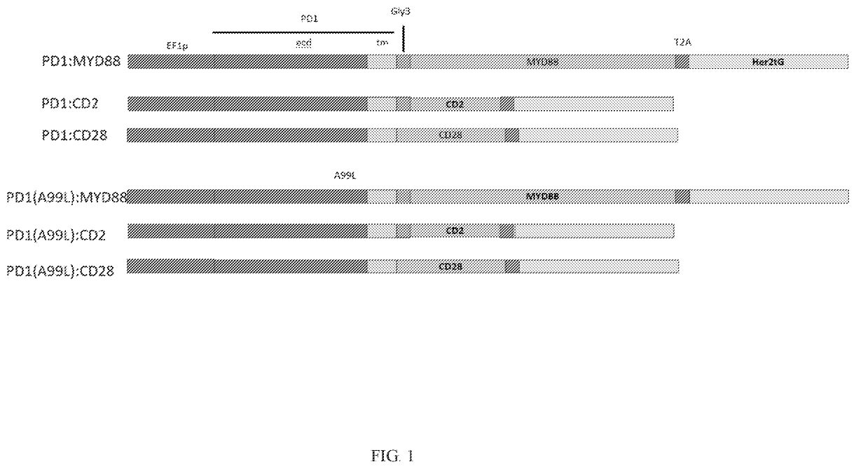

Polynucleotides encoding PD1 chimeric polypeptides were constructed. FIG. 1 depicts certain polynucleotides which encoded: an EF1α promoter; a PD1 extracellular domain (wild-type, or with A99L substitution); a PD1 transmembrane domain; an optional linker (GLY3); an intracellular domain (MYD88 domain, CD2 domain, or CD28 domain); a T2A skip sequence; and a Her2tG selectable marker. Polynucleotides were inserted into a lentivirus expression vector, epHIV7.2. TABLE 2 lists sequences used to construct the polynucleotides.

### Example 2—In Vitro Activity of PD1 Chimeric Polypeptides

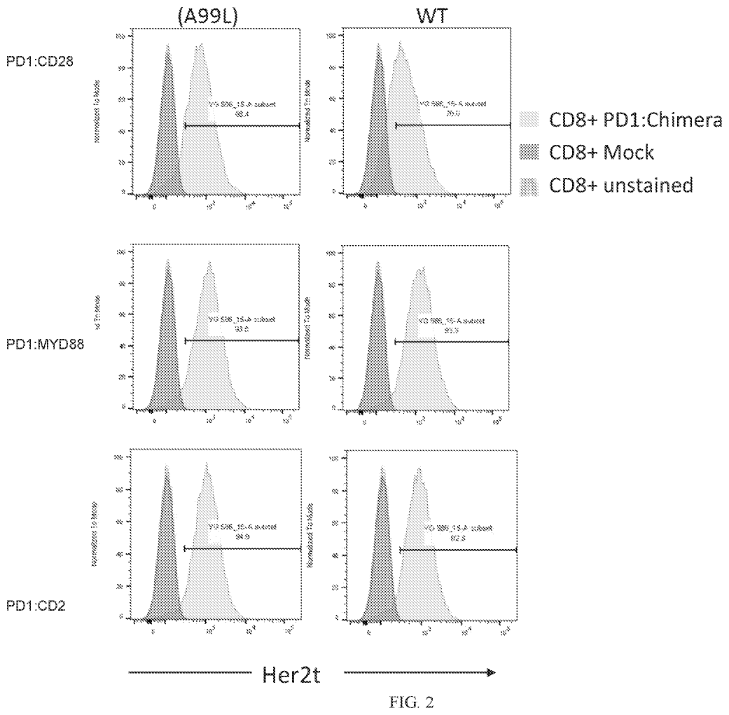

Nucleic acids encoding a certain PD1 chimeric polypeptide and a truncated HER2 (Her2tG) polypeptide marker were introduced into CD8+ T cells. Cells expressing the PD1 chimeric polypeptides were analyzed by flow cytometry. FIG. 2 depicts a flow cytometry analysis of CD8+ T cells for a Her2tG marker co-expressed with a certain PD1 chimeric polypeptide including: a PD1(A99L):CD28 polypeptide; a PD1(wild type):CD28 polypeptide; a PD1(A99L):MYD88 polypeptide; a PD1(wild type):MYD88 polypeptide; a PD1(A99L):CD2 polypeptide; or a PD1(wild type):CD2 polypeptide. All CD8+ T cells had a high positivity for expressed PD1 chimeric polypeptides.

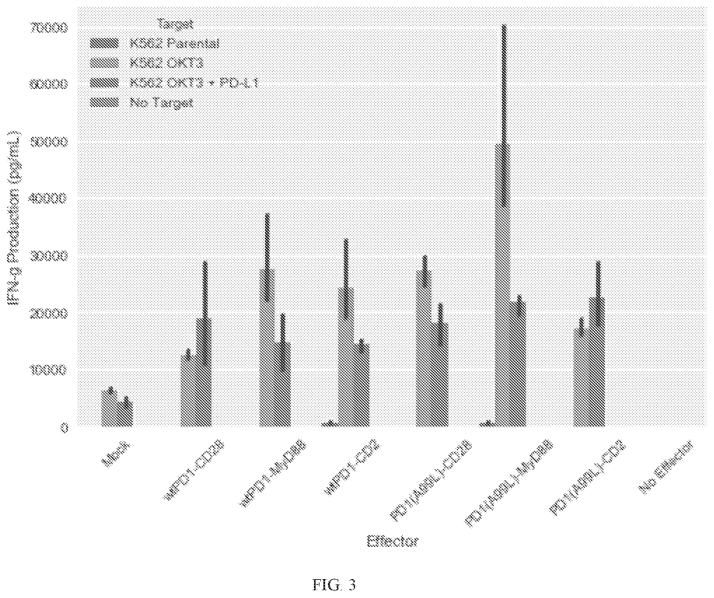

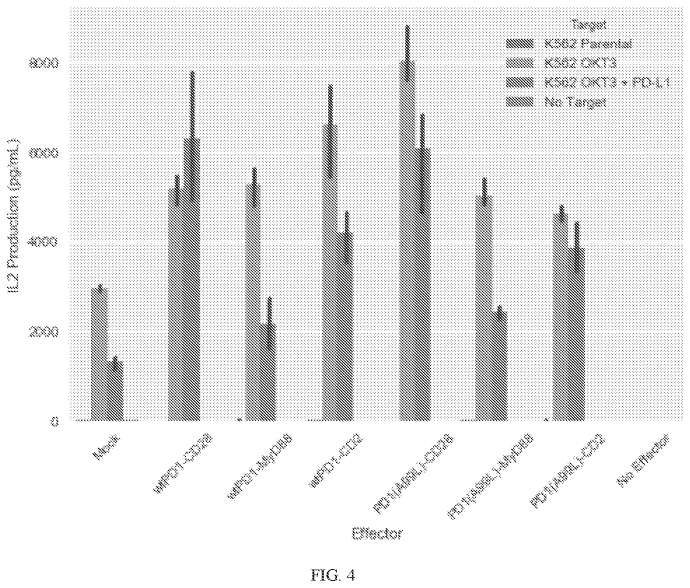

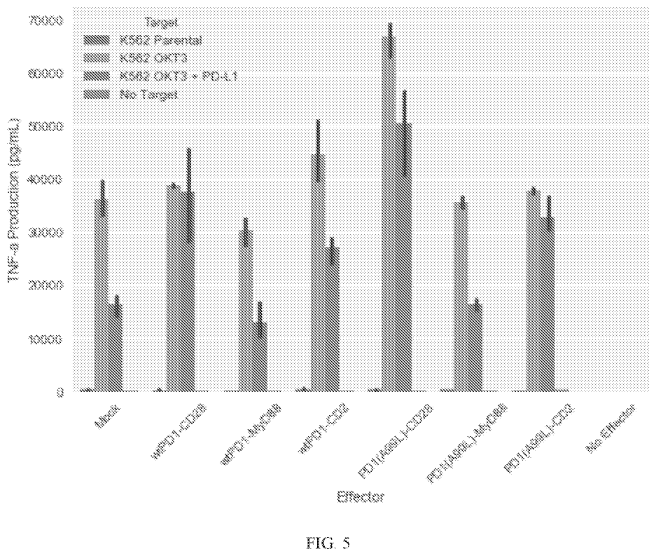

Cytokine release activities for CD8+ T cells containing PD1 chimeric polypeptides were determined in cytokine release assays. Effector CD8+ T cells containing PD1 chimeric polypeptides were incubated with target cells and production of interferon gamma (IFN-g), interleukin-2 (IL-2), or tumor necrosis factor-α (TNF-α) was measured. Target cells includes K562 parental cells, K562 cells overexpressing OKT3, and K562 cells overexpressing OKT3 and PD-L1. With regard to IGN-g production, CD8+ T cells containing the PD1(A99L):MyD88 chimeric polypeptide, or the PD1(A99L):CD2 polypeptide incubated with K562 cells overexpressing OKT3 and PD-L1 had higher levels of IFN-g production compared to CD8+ T cells containing other chimeric polypeptides (FIG. 3). With regard to IL-2 production, CD8+ T cells containing corresponding chimeric polypeptides comprising either a PD1(WT) or a PD1(A99L) domain had similar levels of IL-2 production when incubated with K562 cells overexpressing OKT3 and PD-L1 (FIG. 4). With regard to TNF-α production, CD8+ T cells containing chimeric polypeptides comprising a PD1(A99L) domain had higher levels of TNF-α production than corresponding chimeric polypeptides containing a PD1(WT) domain, when incubated with K562 cells overexpressing OKT3 and PD-L1 (FIG. 5).

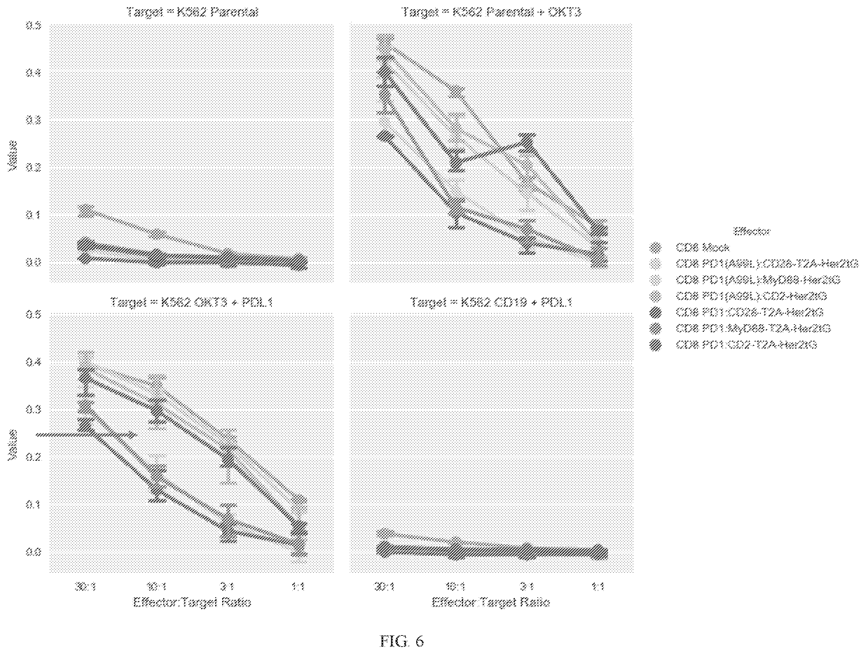

Specific lysis activity of CD8+ T cells containing PD1 chimeric polypeptides was determined using a chromium release assay. Effector CD8+ T cells containing PD1 chimeric polypeptides were incubated at various ratios with target cells including K562 parental cells, K562 cells overexpressing OKT3, K562 cells overexpressing OKT3 and PD-L1, and K562 cells overexpressing CD19 and PD-L1. FIG. 6 depicts line graphs summarizing results of the assay for specific lysis of target cells and includes controls.

### Example 3-In Vitro Activity of CD8+ T Cells Containing a PD1 Chimeric Polypeptide and a CAR

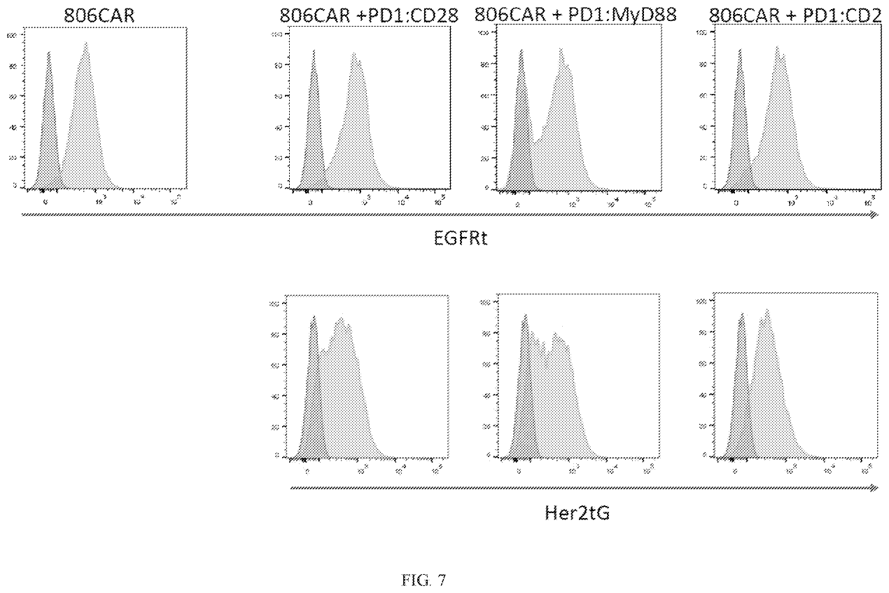

Activities of cells containing both a PD1 chimeric polypeptide and a chimeric antigen receptor (CAR) were determined. An 806 CAR which specifically binds EGFRvIII was used. Nucleic acids encoding (1) a PD1 chimeric polypeptide and a truncated HER2 (Her2tG) selectable marker polypeptide; and (2) an 806 chimeric antigen receptor (CAR) and a truncated EGFR (EGFRt) selectable marker polypeptide, were transduced into CD8+ T cells. A lentivirus vector contained the nucleic acid encoding the 806 CAR. Populations of cells were enriched for transduced and expressing cells using the Her2tG and EGFRt markers. Cells expressing the PD1 chimeric polypeptides and the 806 CAR were analyzed by flow cytometry. FIG. 7 depicts a flow cytometry analysis of CD8+ T cells for the Her2tG marker co-expressed with a PD1 chimeric polypeptide, and for the EGFRt marker co-expressed with the 806 CAR The cells contained the 806 CAR, and either a PD1(A99L):CD28 polypeptide; a PD1(A99L):MYD88 polypeptide; or a PD1(A99L):CD2 polypeptide. All CD8+ T cells had a high positivity for expressed PD1 chimeric polypeptides and the 806 CAR.

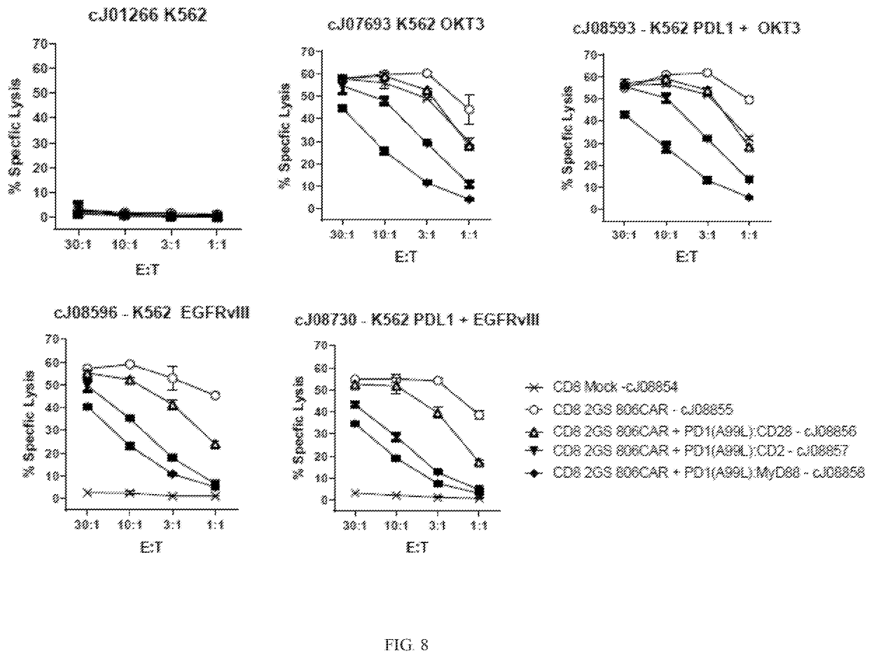

Specific lysis activity of CD8+ T cells containing PD1 chimeric polypeptides and the 806 CAR was determined using a chromium release assay. Effector CD8+ T cells containing the 806 CAR and PD1 chimeric polypeptides were incubated at various ratios with target cells. Target cells included K562 parental cells, K562 cells overexpressing OKT3, K562 cells overexpressing OKT3 and PD-L1, K562 cells overexpressing EGFRvIII, and K562 cells overexpressing EGFRvIII and PD-L1. Effectors were co-incubated with targets at four different ratios 30:1, 10:1, 3:1, and 1:1 over a 4 hour period. FIG. 8 depicts line graphs summarizing results of the assay for specific lysis of target cells and includes controls.

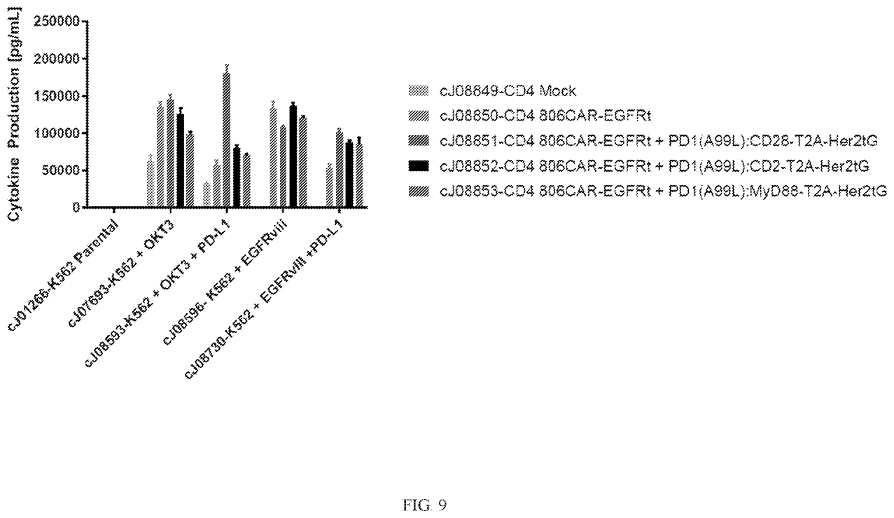

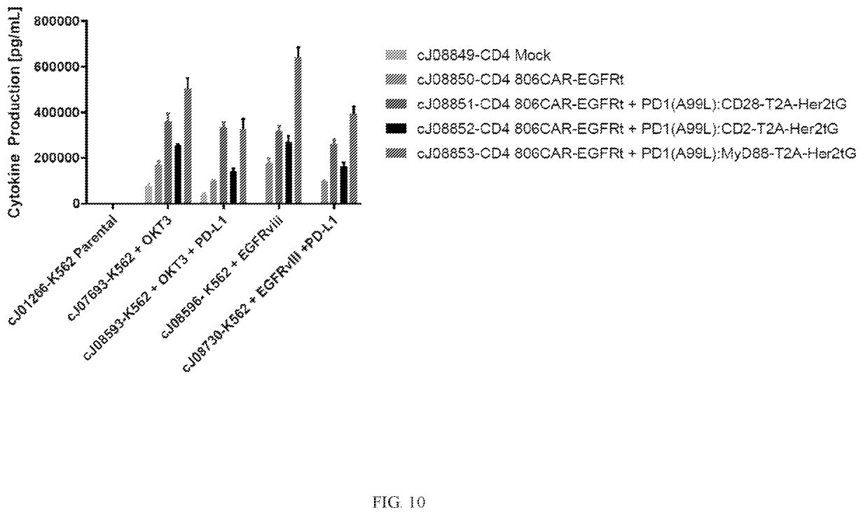

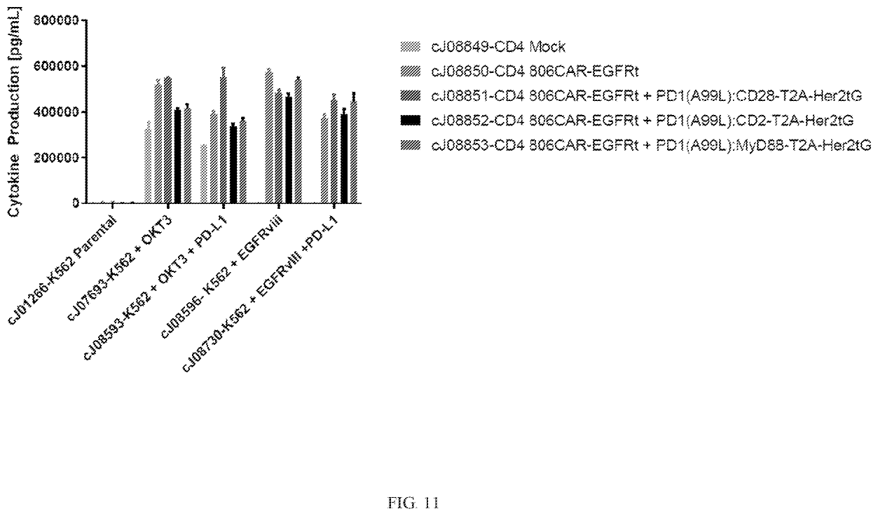

Cytokine release activities for CD8+ T cells containing PD1 chimeric polypeptides were determined in cytokine release assays. Effector CD8+ T cells containing the 806 CAR and PD1 chimeric polypeptides were incubated with target cells and production of IFN-g, IL-2, or TNF-α was measured. Target cells included K562 parental cells, K562 cells overexpressing OKT3, K562 cells overexpressing OKT3 and PD-L1, K562 cells overexpressing EGFRvIII, and K562 cells overexpressing EGFRvIII and PD-L1. FIG. 9 depicts results with regard to IL-2 production. FIG. 11 depicts results with regard to TNF-α production. FIG. 10 depicts results with regard to IGN-g production, and shows that effector cells containing a PD1 chimeric polypeptide and CAR induced a greater response than effector cells containing CAR only.

### Example 4—In Vitro Activity of H9 Cells Containing a PD1 Chimeric Polypeptide and a CAR

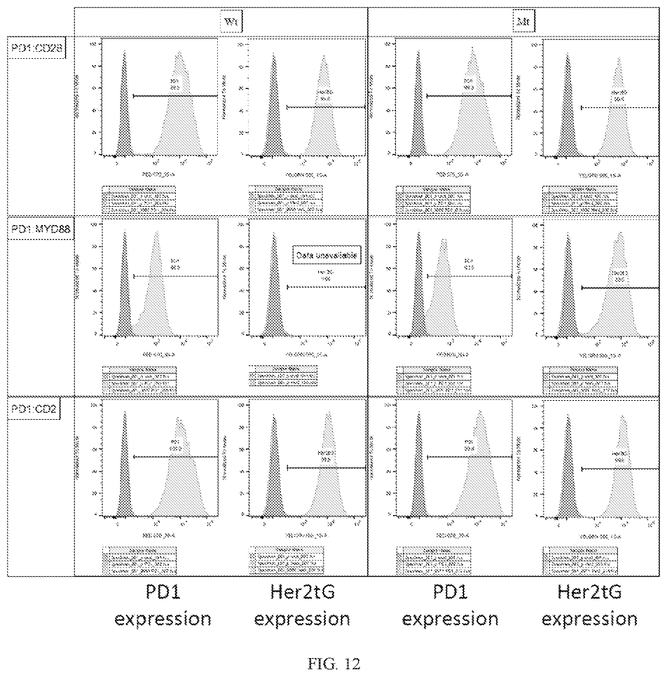

H9 cells were transduced with nucleic acids encoding a 806 CAR linked to a Her2tG selectable marker, and a PD1 chimeric polypeptide. PD1 chimeric polypeptides included: a PD1(A99L):CD28 polypeptide; a PD1(wild type):CD28 polypeptide; a PD1(A99L):MYD88 polypeptide; a PD1(wild type):MYD88 polypeptide; a PD1(A99L):CD2 polypeptide; or a PD1(wild type):CD2 polypeptide. Transduced cells were selected by Her2tG by immunomagnetic selection, then stained for PD1 or Her2tG using specific antibodies. FIG. 12 depicts a FACS analysis of stained cells, and demonstrated that the cells were efficiently selected by Her2tG and express the PD1 chimeric polypeptides.

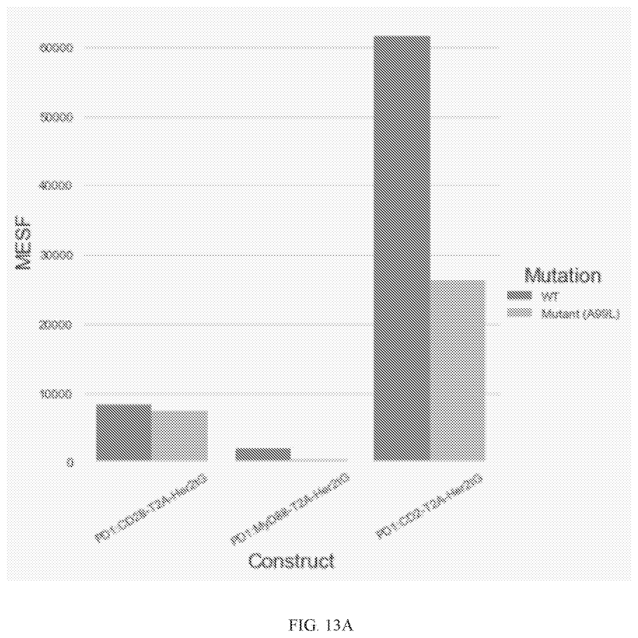

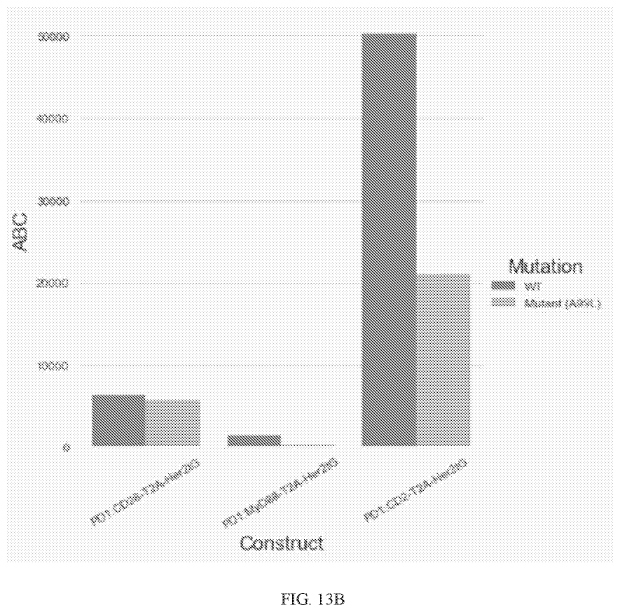

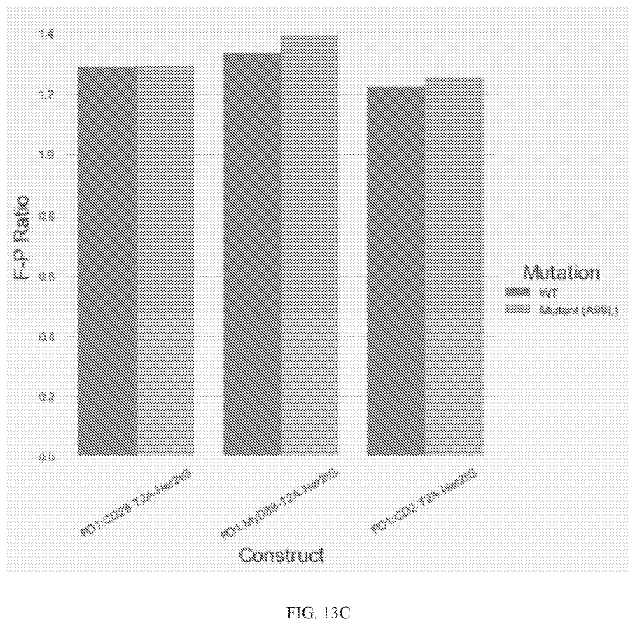

The relative abundance of PD1 chimeric polypeptides on the surface of transduced and selected cells was determined. Her2tG-selected H9 cells were stained with anti PD1 antibody and analyzed by flow analysis against MESF beads or cellular beads to quantify total PD1 chimera proteins on the surface of the cells. Levels of expression were taken into account for affinity assays (FIG. 13A). Cells were Stained with anti PD1 antibody and compared against standard curve established using MESF and Quantum Simply Cellular Beads (FIG. 13B). F/P ratio was derived from the MESF/ABC to give the Fluorophores/Primary antibody which were all about 1.2-1.4 (FIG. 13C). Results demonstrated that the PD1:CD2 chimeric polypeptide levels were higher than the other PD1 chimeric polypeptides (FIG. 13A, FIG. 13B, FIG. 13C). This could have been a result of transduction efficiency, Her2tG selection, and/or the amount of incorporated transgenes into the genome.

A competitive binding assay was performed to determine relative binding affinity between PD1 chimeric polypeptides, in particular PD1(WT):CD28 polypeptide, and the PD1(A99L):CD28 polypeptide.

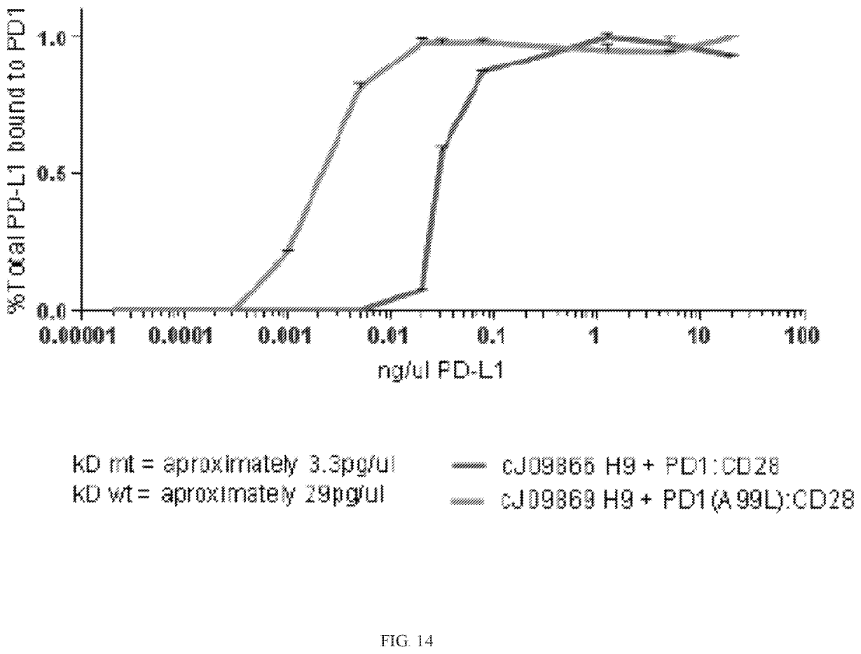

A population of H9 cells containing PD1(WT):CD28 polypeptide were labeled with cell trace violet were mixed with a population of H9 cells containing PD1(A99L):CD28 polypeptide. The cells were then incubated with soluble PD-L1 (0.02 pg/uL to 20 ng/ul) for 30 min and then stained with streptavidin-APC. The two populations were separated out by flow analysis into cell trace violet positive (H9 cells containing PD1(WT):CD28 polypeptide) and cell trace violet negative (H9 cells containing PD1(A99L):CD28 polypeptide) populations. The populations were analyzed for APC positivity. Results demonstrated that the PD1(A99L):CD28 polypeptide had a binding affinity for PD-L1 that was about nine times greater than that for the PD1(WT):CD28 polypeptide (FIG. 14).

### Example 5—Metabolic Analysis of CD8+ T Cells Containing a PD1 Chimeric Polypeptide and a CAR

A seahorse metabolism analysis was performed to determine whether PD1 chimeric polypeptides affect oxygen respiration in CD8+ T cells. CD8+ T cells contained the 806 CAR, and a PD1 chimeric polypeptide. The PD1 chimeric polypeptides included a PD1(A99L):CD28 polypeptide, a PD1(A99L):MYD88, or a PD1(A99L):CD2 polypeptide. After stimulation, the oxygen consumption rate (OCR) for CD8+ cells expressing a PD1 chimera protein was determined. Depending on donor, differences were observed between CD8+806CAR and 806CAR+PD1 chimeras, suggesting the chimeras perturbed the oxygen respiration rate compared to 806CAR alone.

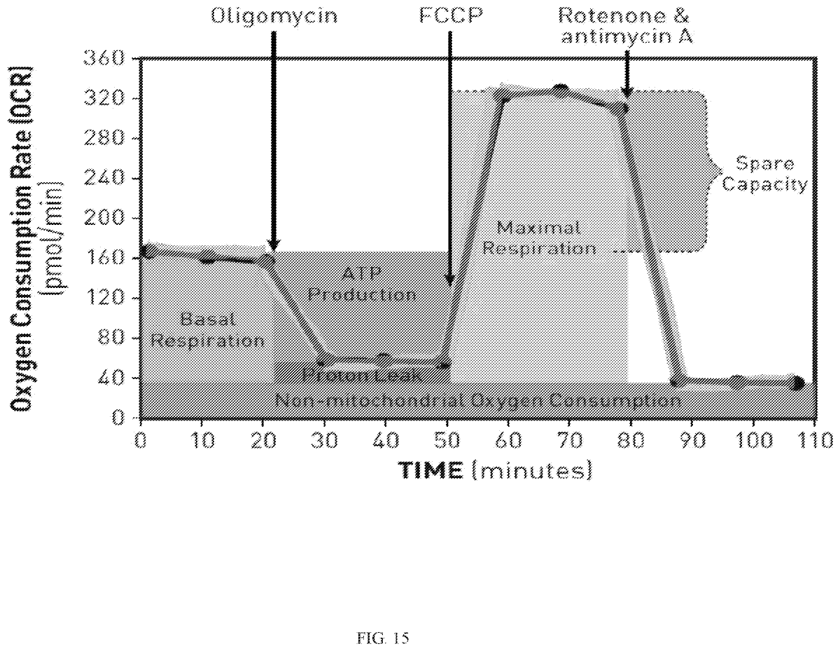

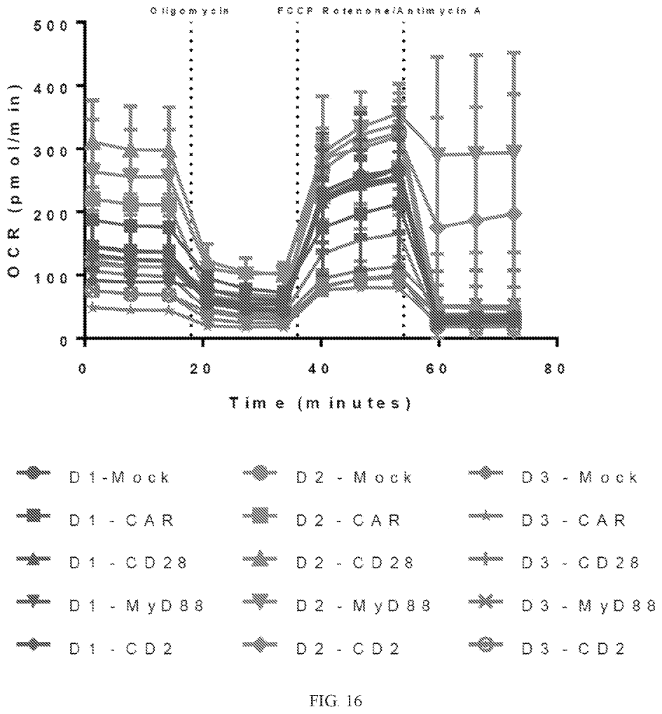

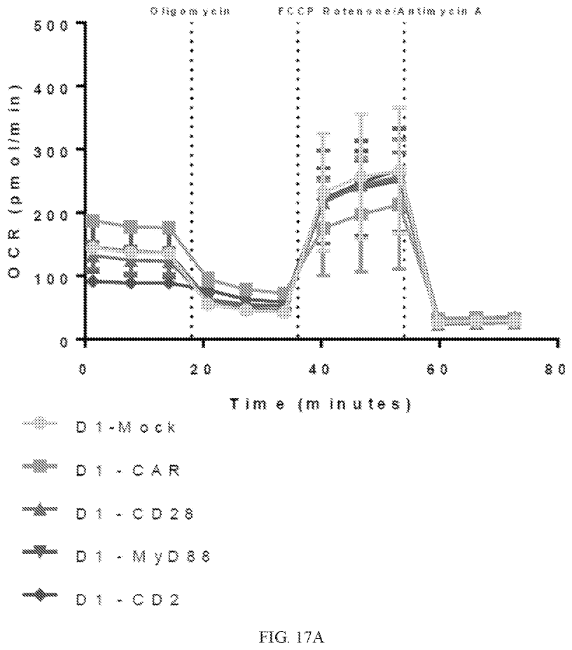

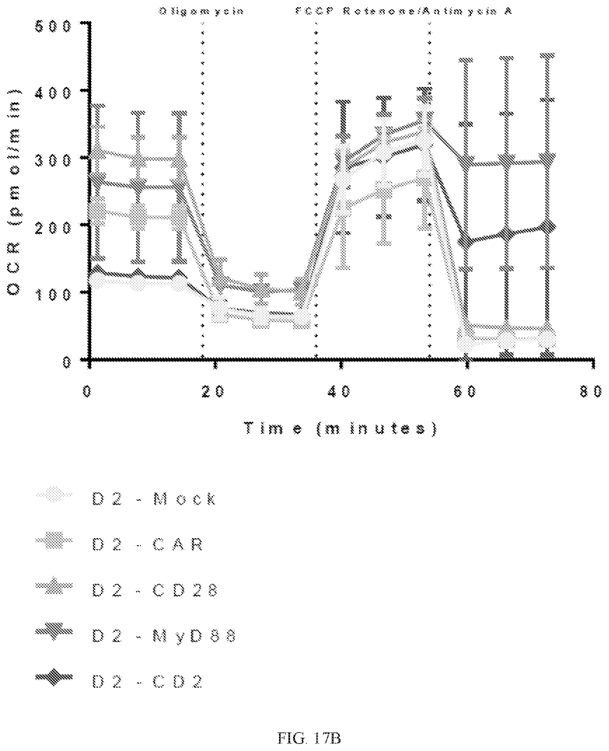

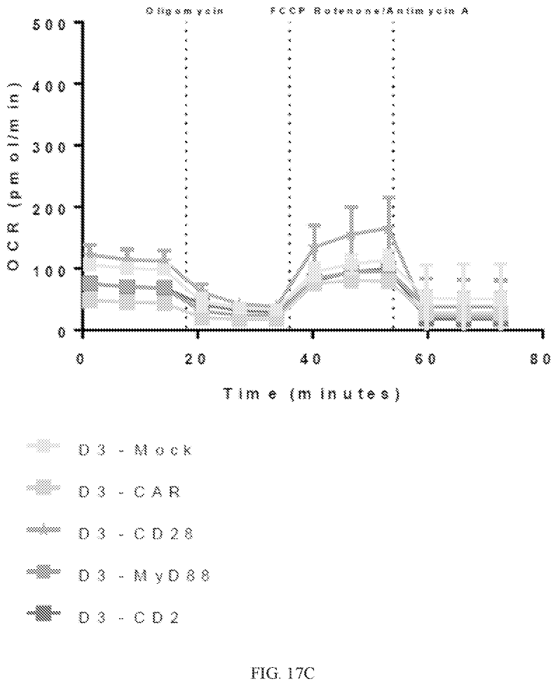

FIG. 15 depicts a seahorse XF cell mitochondria stress test profile. FIG. 16 depicts a graph of oxygen consumption rate for CD8+ T cells from 3 donors (D1, D2, and D3) containing the 806 CAR, and a PD1 chimeric polypeptide. FIG. 17A, FIG. 17B, and FIG. 17C depict a graph of oxygen consumption rate for CD8+ T cells from donors D1, D2, and D3, respectively.

### Example 6—Rank Order of PD1 Chimeric Polypeptides

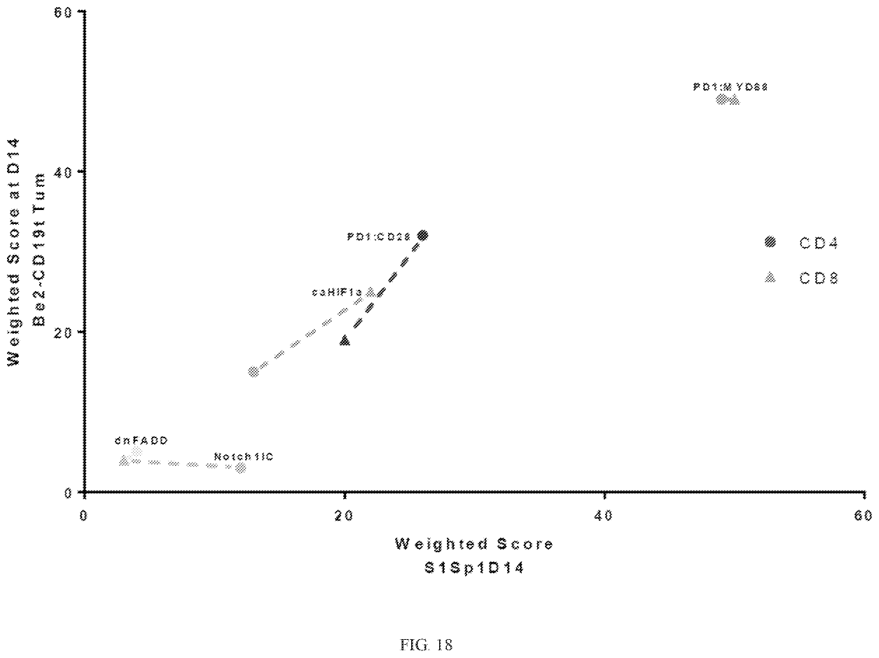

PD1 chimeric transgenes (amongst other transgenes in a library) were introduced into CD4+ or CD8+ T cells along with a CD19CAR though transduction, and were expanded in vitro. To understand whether a PD1 chimera provided a survival or pro-proliferation benefit to the either T cell type, T cells were subjected to an in vivo tumor challenge against a neuroblastoma tumor cell line (Be2) expressing a truncated version of CD19 (CD19t). CD4+ or CD8+ T cells were injected via tail vein. After 14 days, CD4+ or CD8+ T cells were harvested from tumor and isolated using magnetic-activated cell sorting. From T cells that were expanded in vitro (S1Sp1) and subjected to tumor in vivo, genomic DNA was isolated and PD1 chimeric sequences were quantified using droplet digital PCR (ddPCR). A rank order of PD1 chimeric polypeptides in either CD4+ T cells or in CD8+ T cells was determined for in vitro T cell expansion and in vivo tumor challenge analyses. Transgenes were ranked in terms of their abundance and given a score based on their placement after in vitro T cell expansion and in vivo T cell tumor challenge. PD1 chimeric transgenes were consistently abundant throughout in vitro expansion and in vivo tumor challenge suggesting these chimeras have the capacity to provide enhancing features to CD4+ or CD8+ CAR T cells. Donors n=4. FIG. 18 depicts a graph for weighted score at day 14 with regard to ‘Be2-CD19t tumor’ and to weighted score for S1Sp1.

### Example 7—Cytokine Release Assays after First and Second Stimulations

CD8 effector T cells were prepared containing either an anti-CD19 CAR (EF1a_CD19CAR) alone; or the anti-CD19 CAR and a PD1 chimeric polypeptide (iSynPro_PD1(A99L):Chimera). The anti-CD19 CAR was under control of a constitutive promoter, EF1a; the PD1 chimeric polypeptides were under control of an inducible synthetic promoter, iSynPro. The PD1 chimeric polypeptides included: PD1(A99L):CD28; PD1(A99L):MYD88; PD1(A99L):CD2. A neuroblastoma target cell line (Be2-CD19t) was engineered to express a truncated CD19 polypeptide.

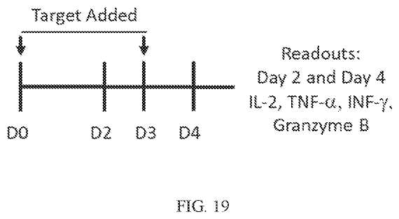

Effector cells were cocultured with target cells at different ratios including: 4:0, 4:1, 2:1, or 1:1. Target cells were added to the coculture at day 0 and day 3 (FIG. 19). Levels of IL-2, TNF, IFN-g and Granzyme B in culture supernatants were measured using Qbeads at day 2 and day 4. Data was collected using the Que3 and analysis was performed using ForeCyt.

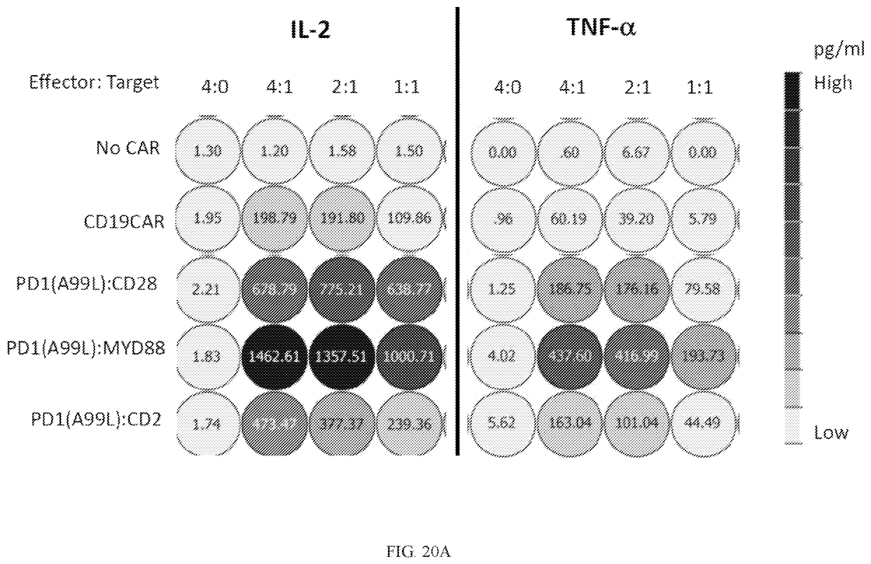

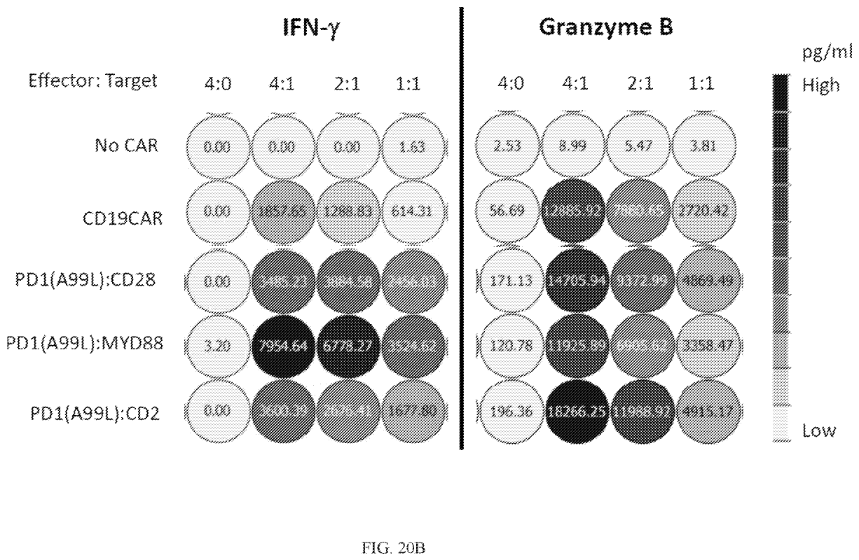

At day 2, the levels of IL2, TNF, IFN-g were elevated for all effector lines expressing a PD1:Chimera compared to CD19CAR alone (FIG. 20A, FIG. 20B). Within the PD1 chimera group, PD1(A99L):MYD88, released the greatest amount of IL-2, TNFa and IFN-g. PD1(A99L):CD2 expressed the highest levels of Granzyme B. Taken together, these data suggested that PD1 chimera release superior effector related cytokine compared to CAR alone when challenged with the Be2 target cell line.

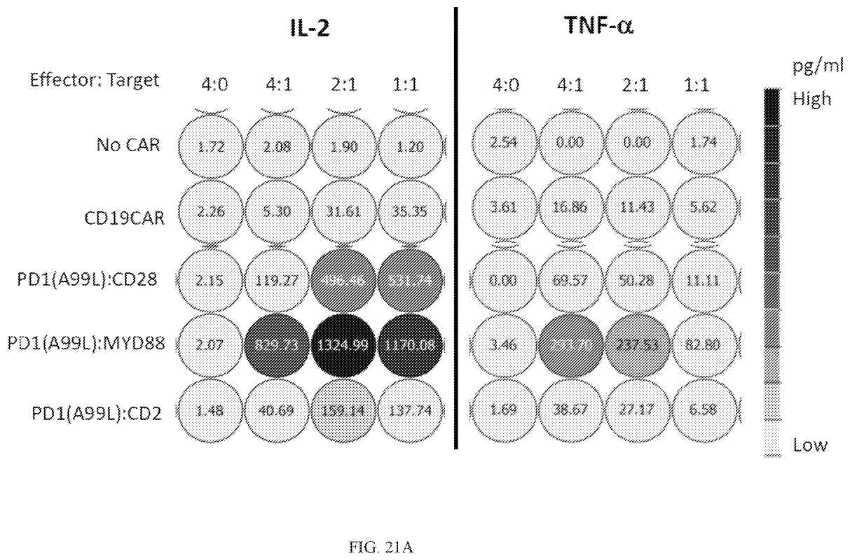

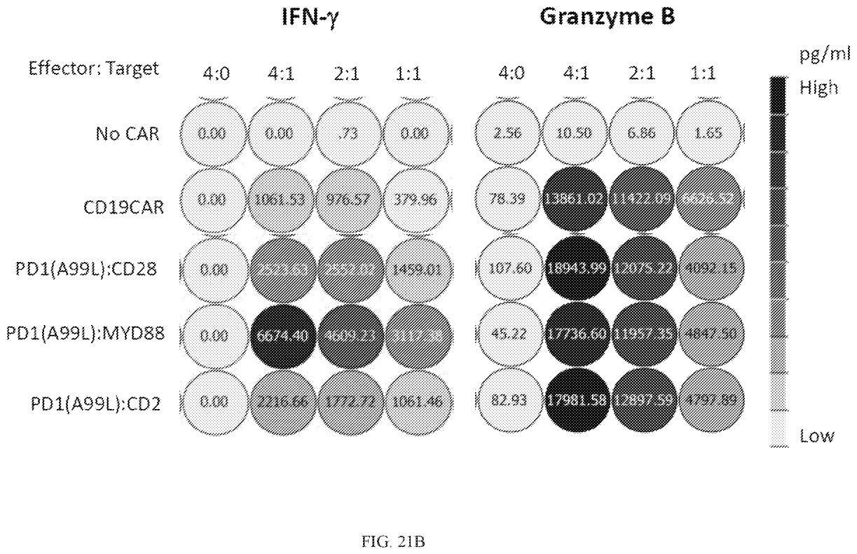

At day 3, effectors were rechallenged and cytokine levels measured at day 4 (FIG. 21A, FIG. 21B). In comparison to levels at day 2 after the initial stimulation at day, cytokines levels were depressed. However, IL2, TNF, IFN-g were elevated for all effector lines expressing a PD1:Chimera compared to CD19CAR alone. Within the PD1 chimera group the same trend was observed where the PD1(A99L):MYD88 resulted in the greatest amount of IL-2, TNFa and IFN-g. Only at the highest E:T ratio were levels of Granzyme B higher than CD19CAR, yet there was no appreciable difference between Granzyme B concentrations within the PD1 chimera set. Taken together, these data suggest that CD8+CD19CAR T cells expressing a regulated version of the transgene PD1(A99L):MYD88 exhibits a superior resistance to decreased cytokine production after recursive stimulation with targets.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A polynucleotide encoding a chimeric polypeptide comprising:
an extracellular PD1 domain; and
an intracellular immunostimulatory domain.

2-69. (canceled)
